(19) United States
(12) Patent Application Publication
(10) Pub. No.: US 2017/0173113 A1
(43) Pub. Date: Jun. 22, 2017

(54) METHODS OF DELIVERING HEPARIN BINDING EPIDERMAL GROWTH FACTOR USING STEM CELL GENERATED EXOSOMES

(71) Applicant: RESEARCH INSTITUTE AT NATIONWIDE CHILDREN'S HOSPITAL, Columbus, OH (US)

(72) Inventors: Gail E. Besner, Dublin, OH (US); Yu Zhou, Dublin, OH (US)

(21) Appl. No.: 15/123,892

(22) PCT Filed: Mar. 13, 2015
(86) PCT No.: PCT/US15/20419

§ 371 Date: Sep. 6, 2016

Related U.S. Application Data
(60) Provisional application No. 61/952,632, filed on Mar. 13, 2014.

(51) Int. Cl. A61K 38/18; A61K 9/107; A61K 35/28

(52) U.S. Cl. CPC A61K 38/1808; A61K 35/28; A61K 9/1075; A61K 2121/00

(57) ABSTRACT

The invention provides for methods of delivering heparin binding epidermal growth factor (HB-EGF) to sites of intestinal injury using stem cell derived exosomes In particular, the invention provides for methods of delivering HB-EGF loaded stem cell-derived exosomes to sites of intestinal injury and methods of protecting a subject and method of treating intestinal injury, such as necrotizing enterocolitis (NEC).

This application claims priority benefit from U.S. Provisional Patent Application No. 61/952,632 filed Mar. 13, 2014, which is incorporated by reference herein in its entirety.

### FIELD OF INVENTION

The invention provides for methods of delivering heparin binding epidermal growth factor (HB-EGF) to sites of intestinal injury using stem cell-derived exosomes. In particular, the invention provides for methods of delivering HB-EGF loaded stem cell-derived exosomes to sites of intestinal injury and methods of protecting a subject from intestinal injury and methods of treating intestinal injury, such as necrotizing enterocolitis (NEC).

### BACKGROUND

Heparin-binding epidermal growth factor (HB-EGF) was first identified in the conditioned medium of cultured human macrophages (Besner et al., Growth Factors, 7: 289-296 (1992), and later found to be a member of the epidermal growth factor (EGF) family of growth factors (Higashiyama et al., Science. 251:936-9, 1991). It is synthesized as a transmembrane, biologically active precursor protein (proHB-EGF) composed of 208 amino acids, which is enzymatically cleaved by matrix metalloproteinases (MMPs) to yield a 14-20 kDa soluble growth factor (sHB-EGF). Pro-HB-EGF can form complexes with other membrane proteins including CD9 and integrin α3β1; these binding interactions function to enhance the biological activity of pro-HB-EGF. ProHB-EGF is a juxtacrine factor that can regulate the function of adjacent cells through its engagement of cell surface receptor molecules.

sHB-EGF is a potent mitogenic and chemoattractant protein for many types of cells. Similar to all members of the EGF family, HB-EGF binds to the “classic” or prototypic epidermal growth factor receptor (EGFR; ErbB-1). However, while the mitogenic function of sHB-EGF is mediated through activation of ErbB-1, its migration-inducing function involves the activation of ErbB-4 and the more recently described N-arginine dibasic convertase (NRDc, Nardilysin). This is in distinction to other EGF family members such as EGF itself, transforming growth factor (TGF)-α and amphiregulin (AR), which exert their signal-transducing effects via interaction with ErbB-1 only. In fact, the NRDc receptor is totally HB-EGF-specific. In addition, unlike most members of the EGF family, which are non-heparin binding, sHB-EGF is able to bind to cell-surface heparin-like molecules (heparan sulfate proteoglycans; HSPG), which act as low affinity, high capacity receptors for HB-EGF. The differing affinities of EGF family members for the different EGFR subtypes and for HSPG may confer different functional capabilities to these molecules in vivo. The combined interactions of HB-EGF with HSPG and ErbB-1/ErbB-4/NRDc may confer a functional advantage to this growth factor. Importantly, endogenous HB-EGF is protective in various pathologic conditions and plays a pivotal role in mediating the earliest cellular responses to proliferative stimuli and cellular injury.

Although the HB-EGF gene is widely expressed, the basal level of its mRNA is relatively low in normal cells. Expression of HB-EGF is significantly increased in response to tissue damage, hypoxia and oxidative stress, and also during wound healing and regeneration. This pattern of expression is consistent with a pivotal role for HB-EGF in ischemia/reperfusion (I/R) injury, regeneration, and repair processes.

Intestinal barrier function represents a critical initial defense against noxious intraluminal substances. Although the intestine is not as essential as the vital organs in the immediate preservation of life, intestinal I/R is as lethal as extensive heart and brain ischemia. The gut has a higher critical oxygen requirement compared to the whole body and other vital organs. Accordingly, the intestinal mucosa is extremely susceptible to I/R and even short periods of ischemia can initiate local and remote tissue damage as well as systemic hemodynamic disturbances.

Reactive oxygen species (ROS), pro-inflammatory cytokines, leukocyte adhesion, and complement activation can all mediate intestinal I/R. Loss of immune and barrier functions of the gut secondary to I/R leads to significant detrimental effects on other organs such as lungs, liver, kidneys and heart, and may result in multiple organ dysfunction syndrome (MODS) and death. Exploring the potential of new therapeutic strategies to enhance the regenerative capacity and/or increase the resistance of the intestine to I/R injury would improve outcome in these patients.

Administration of EGF to prevent tissue damage after an ischemic event in the brains of gerbils has been reported in U.S. Pat. No. 5,057,494 issued Oct. 15, 1991 to Sheffield. The patent projects that EGF “analogs” having greater than 50% homology to EGF may also be useful in preventing tissue damage and that treatment of damage in myocardial tissue, renal tissue, spleen tissue, intestinal tissue, and lung tissue with EGF or EGF analogs may be indicated. However, the patent includes no experimental data supporting such projections.

EGF family members are of interest as intestinal protective agents due to their roles in gut maturation and function. Infants with NEC have decreased levels of salivary EGF, as do very premature infants (Shin et al., J Pediatr Surg 35:173-176, 2000; Warner et al., J Pediatr 150:358-6, 2007). Studies have demonstrated the importance of EGF in preserving gut barrier function, increasing intestinal enzyme activity, and improving nutrient transport (Warner et al., Semin Pediatr Surg 14:175-80, 2005). EGF receptor (EGFR) knockout mice develop epithelial cell abnormalities and hemorrhagic necrosis of the intestine similar to neonatal necrotizing enterocolitis (NEC), suggesting that lack of EGFR stimulation may play a role in the development of NEC (Miettinen et al., Nature 376:337-41, 1995). Dvorak et al. have shown that EGF supplementation reduces the incidence of experimental NEC in rats, in part by reducing apoptosis, barrier failure, and hepatic dysfunction (Am J Physiol Gastrointest Liver Physiol 282:G156-G164, 2002). Vinter-Jensen et al., investigated the effect of subcutaneously administered EGF (150 μg/kg/12 hours) in rats, for 1, 2 and 4 weeks, and found that EGF induced growth of small intestinal mucosa and muscularis in a time-dependent manner (Regul Pept 61:135-142, 1996). Several case reports of clinical administration of EGF also exist. Sigalet et al. administered EGF (100 μg/kg/day) mixed with enteral feeds for 6 weeks to pediatric patients with short bowel syndrome (SBS), and reported improved nutrient absorption and increased tolerance to enteral feeds with no adverse effects (J Pediatr Surg 40:763-8, 2005). Sullivan et al., in a prospective, double-blind, randomized controlled study that included 8 neonates with NEC, compared the effects of a 6-day continuous intravenous infusion of EGF (100 ng/kg/hour) to placebo, and found a positive trophic effect of EGF on the intestinal mucosa (Ped Surg 42:462-469, 2007). Palomino et al. examined the efficacy of EGF in the treatment of duodenal ulcers in a multicenter, randomized, double blind human clinical trial in adults. Oral human recombinant EGF (50 mg/ml every 8 h for 6 weeks) was effective in the treatment of duodenal ulcers with no side effects noted (Scand J Gastroenterol 35:1016-22, 2000).

Enteral administration of E. coli-derived HB-EGF has been shown to decrease the incidence and severity of intestinal injury in a neonatal rat model of NEC, with the greatest protective effects found at doses of 600 or 800 μg/kg/dose (Feng et al., Semin Pediatr Surg 14:167-74, 2005). In addition, HB-EGF is known to protect the intestines from injury after intestinal ischemia/reperfusion injury (El-Assal et al., Semin Pediatr Surg 13:2-10, 2004) or hemorrhagic shock and resuscitation (El-Assal et al., Surgery 142:234-42, 2007).

Mesenchymal stem cells (MSC) have the ability to differentiate into different cell lineages and can stimulate wound healing via paracrine signaling pathways. Preclinical studies have shown that MSC can regulate the host immune response, thus avoiding recognition and subsequent rejection by recipients. The robust, self-renewing, multilineage differentiation potential of MSC makes these cells very desirable candidates for possible clinical cellular therapy. Baksh et al., J Cell Mol Med 2004; 8(3):301-16. Ongoing investigations are exploring ways to optimize MSC efficacy prior to therapeutic delivery, including preconditioning by exposure to hypoxia, Hu et al., J Thorac Cardiovasc Surg 2008; 135(4):799-808, growth factors, Hahn et al., J Am Coll Cardiol 2008; 51(9):933-43, and various cytokines. Gui et al., Mol Cell Biochem 2007; 305(1-2):171-8, Pasha et al., Cardiovasc Res 2008; 77(1):134-42, Liu et al., Acta Pharmacol Sin 2008; 29(7):815-22.

Local stem cell (SC) delivery may result in increased risks and side effects including bleeding and tissue injury when administered by direct intralesional injection, and occlusion and embolization when administered intra-arterially to target organs Dimmeler et al., Arterioscler Thromb Vasc Biol. 2008; 28:208-216, Ott et al., Basic Res Cardiol. 2005; 100:504-517, Sherman et al., Nat Clin Pract Cardiovasc Med. 2006; 3 Suppl 1:S57-64. Intravenous (IV) infusion has been used for systemic SC delivery in preclinical studies Lee et al., Cell Stem Cell. 2009; 5:54-63, Abdel-Mageed et al., Blood. 2009; 113:1201-1203, and in clinical trials Lazarus et al., Bone Marrow Transplant. 1995; 16:557-564, Horwitz et al., Nat Med. 1999; 5:309-313, Le Blanc et al., Lancet. 2008; 371:1579-1586, Wu et al., Transplantation. 2011; 91:1412-1416, in the past two decades. However, it has been noted that a large fraction of systemically infused MSC become trapped in the lung due to their large size Schrepfer et al., Transplant Proc. 2007; 39:573-576. Thus, pulmonary passage is a major obstacle for IV stem cell delivery, which is termed the “pulmonary first-pass effect” Fischer et al., Stem Cells Dev. 2009; 18:683-692. This effect not only causes poor efficiency of MSC delivery and decreased specific homing of the cells, but it also threatens the life of experimental animals Ramot et al., Nanotoxicology. 2010; 4:98-105. Pulmonary sequestration by MSC intravascular infusion causes death in small adult animals, with the mortality rate ranging from 25% to 40%.

It is known that mesenchymal stem cell (MSC) and neural stem cells (NSC) protect the intestines from NEC. While stem cell therapy for injured organs was initially based on the hypothesis that stem cell engraft in injured tissues and then differentiate into cells that replace damaged cells, engraftment and differentiation may not account for all of their therapeutic effects. In addition, the fact that a large portion of SC death occurs within the first week after transplantation due to the host hostile environment limits the application of SC to a variety of diseases. Some studies show that less than 1% of transplanted MSC migrate to the injury site, with most trapped in liver, spleen and lungs. On the other hand, stem cell may exert their effects via the release of secreted components since treatment response is often greater than can be accounted for by engraftment efficiency and administration of amniotic fluid derived-MSC conditioned medium protects the intestines from NEC.

The prevention and treatment of ischemic damage in the clinical setting continues to be a challenge in medicine. Exosomes are nanosized microvesicles for mediating stem cell signaling because they are involved in cellular exchange of a complex cargo that is protected from the harsh and degrading conditions of the extracellular environment. These characteristics make exosomes a potential non-cell based therapy to treat NEC and other intestinal injuries such as those related to hemorrhagic shock, and ischemia and inflammatory conditions.

### SUMMARY OF INVENTION

Factors that protect the intestine from injury and promote early intestinal healing by restitution may significantly improve the clinical outcome of human subjects suffering many forms of intestinal injury. HB-EGF has previously been demonstrated to have potent mitogenic activity for a variety of cell types, including smooth muscle cells, epithelial cells, fibroblasts, keratinocytes and renal tubular cells, and is a known chemotactic agent for various cell types. Furthermore, mesenchymal stem cells are an attractive target for regenerative medicine. Their properties in cell culture and their in vitro behavior are becoming increasingly characterized.

In the past, it was demonstrated that neural stem cell (NSC) transplantation protects the enteric nervous system (ENS) during experimental necrotizing enterocolitis (NEC), but it is unclear whether SC engraftment or SC-secreted products mediate these effects. The invention provide for stem cell-secreted exosomes (lipid membrane vesicles of 50-150 nm diameter that carry microRNAs, mRNA and proteins) that specifically target injured intestinal neurons and protect the ENS from NEC-induced injury. The experiments provided herein describe tagged exosomes from enteric NSC and demonstrate that these exosomes can be administered intraperitoneally (IP) to rat pups exposed to NEC. In vitro, exosomes were applied to mixed cultures of intestinal cells subjected to anoxic injury.

The NSC-derived exosomes were characterized as double membrane encapsulated microvesicles with a diameter of 30-150 nm. Exosomes administered IP homed to injured intestinal segments in pups exposed to NEC, leading to significantly decreased mortality (39% vs. 70%, p<0.05), and ameliorated intestinal histologic injury. In vitro, NSC-derived exosomes specifically targeted NSC and injured neurons, and reduced neuronal apoptosis after anoxic injury. These data show that NSC-derived exosomes can protect the ENS from injury during NEC, suggesting that they mediate the therapeutic efficacy of NSCs. The invention provides for methods of treating intestinal injury using non-cell based therapies to protect the ENS from injury during NEC in the future.

The data provided herein supports a role for secreted nanovesicles in mediating paracrine effects of SC in tissue repair, and highlight the importance of understanding their biological function and potential clinical utility. The ability of HB-EGF to protect SC plays a major role in its intestinal cytoprotective effects, and that at least some of the beneficial effects of SC are mediated by SC-secreted exosomes. The positive impact of this innovative approach is decreased NEC-related morbidity and mortality with substantive improvements in clinical outcomes.

HB-EGF is known to be present in human amniotic fluid and breast milk, ensuring continuous exposure of the fetal and newborn intestine to endogenous levels of the growth factor (Michalsky et al., J Pediatr Surg 37:1-6, 2006). Thus, the developing fetus and the breastfed newborn are continually exposed to HB-EGF naturally both before and after birth. Supplementation of enteral feeds with a biologically active substance such as HB-EGF, to which the fetus and newborn are naturally exposed, may represent a logical and safe way to reduce intestinal injury resulting in NEC. HB-EGF supplementation of feeds in very low birth weight patients (<1500 g) who are most at risk for developing NEC is contemplated to facilitate maturation, enhance regenerative capacity, and increase the resistance of the intestinal mucosa to injury.

Intragastric administration of HB-EGF to rats is known to lead to delivery of the growth factor to the entire GI tract including the colon within 8 hours. HB-EGF is excreted in the bile and urine after intragastric or intravenous administration (Feng et al., Peptides. 27(6):1589-96, 2006). In addition, intragastric administration of HB-EGF to neonatal rats and minipigs has no systemic absorption of the growth factor (unpublished data). These findings collectively support the clinical feasibility and safety of enteral administration of HB-EGF in protection of the intestines from injury.

The invention provides stem cell derived exosomes comprising heparin binding epidermal growth factor (HB-EGF) product or a fragment thereof wherein the exosomes are produced by a stem cell transfected to express HB-EGF product or a fragment thereof. The stem cell-derived exosomes of the invention may be derived from any type of stem cells including neural stem cells, mesenchymal stem cells (MSC), intestinal stem cells (ISC), neural stem cells (NSC) or embryonic stem cells (ESC). Further, HB-EGF product expressed by the stem cell comprises amino acids of 74-148 of SEQ ID NO: 2. The invention also provides for compositions comprising the SC-derived exosomes of the invention and a carrier such as pharmaceutical compositions comprising the SC-derived exosomes of the invention and a pharmaceutically acceptable carrier.

The invention also provides for methods of delivering a HB-EGF product or a fragment thereof to a site of intestinal injury comprising administering stem cell-derived exosomes comprising HB-EGF to a subject suffering from an intestinal injury.

The invention also provides for use of the stem cell-derived exosomes for the preparation of a medicament for delivering HB-EGF product or fragment thereof to the site of intestinal injury wherein the stem cell derived exosomes comprise HB-EGF product or a fragment thereof. The invention also provides for stem cell-derived exosomes for use in delivering a HB-EGF product or fragment thereof to a site of intestinal injury,

In another aspect, the invention provides for methods of treating an intestinal injury comprising administering a stem cell-derived exosomes comprising HB-EGF product or a fragment thereof in an amount effective to reduce the severity of the intestinal injury.

The invention also provides for use of the stem cell-derived exosomes for the preparation of a medicament for treating intestinal injury wherein the stem cell derived exosomes comprise HB-EGF product or a fragment thereof in an amount effective to reduce the severity of the intestinal injury. The invention also provides for stem cell-derived exosomes for use in treating intestinal injury, wherein the stem cell-derived exosomes comprises HB-EGF product or a fragment thereof in an amount effective to reduce the severity of the intestinal injury.

The invention also provides for methods of reducing damage to intestinal tissue in a patient suffering from intestinal injury comprising administering a stem cell-derived exosomes comprising a HB-EGF product or a fragment thereof in an amount effective to protect the intestinal tissue in the patient.

The invention also provides for use of the stem cell-derived exosomes for the preparation of a medicament for reducing damage to intestinal tissue in a patient suffering from intestinal injury wherein the stem cell derived exosomes comprise HB-EGF product or a fragment thereof in an amount effective to reduce damage to intestinal tissue and to protect intestinal tissue in a patient. The invention also provides for stem cell-derived exosomes for use in reducing intestinal damage, wherein the stem cell-derived exosomes comprises HB-EGF product or a fragment thereof in an amount effective to protect the intestinal tissue in a patient suffering from intestinal injury.

The invention also provides for methods of treating an infant to abate NEC, comprising administering stem cell-derived exosomes comprising a HB-EGF product of a fragment thereof in an amount effective to reduce the severity of NEC, including administering somatic stem cells or embryonic stem cells.

The invention also provides for use of the stem cell-derived exosomes for the preparation of a medicament for bating NEC in an infant or treating NEC in an infant wherein the stem cell derived exosomes comprise HB-EGF product or a fragment thereof in an amount effective to abate NEC. The invention also provides for stem cell-derived exosomes for use in abating or treating NEC in an infant, wherein the stem cell-derived exosomes comprises HB-EGF product or a fragment thereof in an amount effective to reduce the severity of NEC.

The invention provides for methods of reducing the risk of developing NEC in an infant, comprising administering stem cell-derived exosomes comprising HB-EGF product or a fragment thereof in an amount effective to reduce the onset of NEC.

The invention also provides for use of the stem cell-derived exosomes for the preparation of a medicament for reducing the risk of developing NEC in an infant, wherein the stem cell derived exosomes comprise HB-EGF product or a fragment thereof in an amount effective to reduce the onset of NEC. The invention also provides for stem cell-derived exosomes for use in reducing the risk of developing NEC, wherein the stem cell-derived exosomes comprises HB-EGF product or a fragment thereof in an amount effective to reduce the onset of NEC.

In any of the methods or uses of the invention, the exosomes may be derived from a neural stem cell. In addition, in any of the methods of the invention, the stem cells are transfected to express HB-EGF or a fragment thereof, for example the HB-EGF product comprises amino acids of 74-148 of SEQ ID NO: 2.

Any type of stem cell may be used to generate the SC-derived exosomes for any of the methods or uses of the invention. For example, any of the methods of the invention may be carried out using exosomes derived from mesenchymal stem cells (MSC), intestinal stem cells (ISC), neural stem cells (NSC) or embryonic stem cells (ESC). In any of the methods of the invention, the intestinal injury may be caused by necrotizing enterocolitis, hemorrhagic shock, resuscitation, ischemia/reperfusion injury, intestinal inflammatory conditions or intestinal infections. Further, in any of the methods of the invention, the subject may be suffering from Hirschprung's Disease, intestinal dysmotility disorders, intestinal pseudo-obstruction (Ogilvie's Syndrome), inflammatory bowel disease, irritable bowel syndrome, radiation enteritis or chronic constipation, Crohn's Disease, bowel cancer, or colorectal cancers. In addition, the in any of the methods of the invention, the exosomes may be administered to an infant patient.

In any of the methods or uses of the invention, the exosomes are administered intravenously or intraperitoneally. Furthermore, in any of the methods of the invention, the exosomes are administered immediately following the intestinal injury or within 1-5 hours following the intestinal injury.

The onset of symptoms of NEC refers to the occurrence or presence of one or more of the following symptoms: temperature instability, lethargy, apnea, bradycardia, poor feeding, increased pregavage residuals, emesis (may be bilious or test positive for occult blood), abdominal distention (mild to marked), occult blood in stool (no fissure), gastrointestinal bleeding (mild bleeding to marked hemorrhaging), significant intestinal distention with ileus, edema in the bowel wall or peritoneal fluid, unchanging or persistent “rigid” bowel loops, pneumatosis intestinalls, portal venous gas, deterioration of vital signs, evidence of septic shock and pneumoperitoneum.

In one embodiment, the invention contemplates administering stem cell-derived exosomes comprising a HB-EGF product to a premature infant. The term “premature infant” (also known as a “premature baby” or a “preemie”) refers to babies born having less than 36 weeks gestation. In another embodiment, the invention provides for methods of administering stem cell-derived exosomes to an infant having a low birth weight or a very low birth weight. A low birth weight is a weight less than 2500 g (5.5 lbs.). A very low birth weight is a weight less than 1500 g (about 3.3 lbs.). The invention also provides for methods of administering stem cell-derived exosomes comprising a HB-EGF product to infants having intrauterine growth retardation, fetal alcohol syndrome, drug dependency, prenatal asphyxia, shock, sepsis, or congenital heart disease.

In addition to a HB-EGF product, the methods of the invention may utilize stem cell-derived exosomes comprising any EGF receptor agonist. An EGF receptor agonist refers to a molecule or compound that activates the EGF receptor or induces the EGF receptor to dimerize, autophosphorylate and initiate cellular signaling. For example, any of the methods of the invention may be carried out with an EGF receptor agonist such as an EGF product or a HB-EGF product.

The methods of the invention are carried out with stem cell-derived exosomes comprising a dose of each of a HB-EGF product that is effective to reduce the onset or severity of intestinal injury or to protect or rejuvenate the intestinal tissue in patient. Exemplary effective doses are 100 μg/kg dose, 105 μg/kg dose, 110 μg/kg dose, 115 μg/kg dose, 120 μg/kg dose, 125 μg/kg dose, 130 μg/kg dose, 135 μg/kg dose, 140 μg/kg dose, 200 μg/kg dose, 250 μg/kg dose, 300 μg/kg dose, 400 μg/kg dose, 500 μg/kg dose, 550 μg/kg dose, 570 μg/kg dose, 600 μg/kg dose, 800 μg/kg dose and 1000 μg/kg dose. Exemplary dosage ranges of a HB-EGF product that is effective to reduce the onset or severity of intestinal injury or to protect or rejuvenate the intestinal tissue in patients are 100-140 μg/kg, 100-110 μg/kg dose, 110-120 μg/kg dose, 120-130 μg/kg dose, 120-140 μg/kg dose and 130-140 μg/kg dose.

For all the methods of the invention, the HB-EGF product is a polypeptide having the amino acid sequence of SEQ ID NO: 2 or a fragment thereof that competes with HB-EGF for binding to the ErbB-1 receptor and has ErbB-1 agonist activity. A preferred HB-EGF product is a fragment of HB-EGF that comprises amino acids of 74-148 of SEQ ID NO: 2 (human HB-EGF(74-148). In addition, the HB-EGF product includes fragments of HB-EGF that induce epithelial cell or somatic stem cell, such as MSC or ISC, proliferation, fragments of HB-EGF that induce epithelial cell or somatic stem cell, such as MSC or ISC, migration, fragments of HB-EGF that promote epithelial cell or somatic stem cell, such as MSC or ISC, viability, and a fragment of HB-EGF that protects epithelial cells or somatic stem cells, such as MSC or ISC form apoptosis or other types of cellular injury.

In preferred embodiments, stem cell-derived exosomes comprising a HB-EGF product are administered in any of the methods of the invention immediately after intestinal injury, or shortly after intestinal injury such as within about 1, about 2, about 3, about 4 or about 5 hours after intestinal injury. However, the invention provides for methods of administering stem cell-derived exosomes comprising a HB-EGF product at any time during or after intestinal injury, such as later than about 5 hours after injury. For example, the invention contemplates administering stem cell-derived exosomes comprising a HB-EGF product to subjects seeking treatment several or many hours after injury or after hemorrhagic shock (HS) or NEC has developed, or in cases where treatment is delayed for some reason.

When the intestinal injury is caused by HS or HS/R, the invention provides method of administering stem cell-derived exosomes comprising a HB-EGF product immediately after HS or HS/R or shortly after HS or HS/R such as within about 1, about 2, about 3, about 4 or about 5 hours after resuscitation. However, the invention provides for methods of administering stem cell-derived exosomes comprising a HB-EGF product at any time during or after HS or HS/R has developed, such as later than about 5 hours after injury or later than about 5 hours after HS or HS/R has developed. For example, the invention contemplates administering stem cell-derived exosomes comprising a HB-EGF product to subjects seeking treatment several or many hours after injury or after HS has developed, or in cases where treatment is delayed for some reason. In addition, it is preferred that the stem cell-derived exosomes comprising HB-EGF product be administered before ischemia, hypoxia or necrotizing enterocolitis takes effect.

When the intestinal injury is NEC, the methods of the invention include administering stem cell-derived exosomes comprising a HB-EGF product, immediately after birth or shortly after birth. For example, the dose may be administered within about the first hour following birth, within about 2 hours following birth stem cell-derived exosomes h, within about 3 hours following birth, within about 4 hours following birth, within about 5 hours following birth, within about 6 hours following birth, within about 7 hours following birth, within about 8 hours following birth, within about 9 hours following birth, within about 10 hours following birth, within about 11 hours following birth, within about 12 hours after birth, within about 13 hours after birth, within about 14 hours after birth, within about 15 hours after birth, within about 16 hours after birth, within about 17 hours after birth, within about 18 hours after birth, within about 19 hours after birth, within about 20 hours after birth, within about 21 hours after birth, within about 22 hours after birth, within about 23 hours after birth, within about 24 hours after birth, within about 36 hours after birth, within about 48 hours after birth or within about 72 hours after birth.

The invention contemplates administering stem cell-derived exosomes comprising a HB-EGF product to an infant suffering or at risk of developing NEC. In one embodiment, stem cell-derived exosomes comprising a HB-EGF product are administered within about the first 12-72 hours after birth. For example, the doses a HB-EGF product are administered about 12 hours after birth, about 24 hours after birth, about 36 hours after birth, about 48 hours after birth or about 72 hours after birth. In further embodiments, the doses are administered between hours 1-4 following birth or between hours 2-5 following birth or between hours 3-6 following birth or between hours 4-7 following birth or between hours 5-8 following birth or between hours 6-9 following birth or between hours 7-10 following birth or between hours 8-11 following birth, between hours 9-12 following birth, between hours 10-13 following birth, between hours 11-14 following birth, between hours 12-15 following birth, between hours 13-16 following birth, between hours 14-17 following birth, between hours 15-18 following birth, between hours 16-19 following birth, between hours 17-20 following birth, between hours 18-21 following birth, between hours 19-22 following birth, between hours 20-23 following birth, between hours 21-24 following birth, between hours 12-48 following birth, between hours 24-36 following birth, between hours 36-48 following birth and between hours 48-72 after birth.

In another embodiment, stem cell-derived exosomes comprising a HB-EGF product are administered within 24 hours following the onset of at least one symptom of NEC, such as administering stem cell-derived exosomes comprising a HB-EGF product within about the first 12-72 hours after onset of at least one symptom of NEC. For example, the doses of stem cell-derived exosomes comprising a HB-EGF product are administered about 12 hours following the occurrence or presence of a symptom of NEC, about 24 hours following the occurrence or presence of a symptom of NEC, about 36 hours following the occurrence or presence of a symptom of NEC, about 48 hours following the occurrence or presence of a symptom of NEC or about 72 hours following the occurrence or presence of a symptom of NEC. In further embodiments, the doses are administered between hours 1-4 following the occurrence or presence of a symptom of NEC or between hours 2-5 following the occurrence or presence of a symptom of NEC or between hours 3-6 following the occurrence or presence of a symptom of NEC or between hours 4-7 following the occurrence or presence of a symptom of NEC or between hours 5-8 following the occurrence or presence of a symptom of NEC or between hours 6-9 following the occurrence or presence of a symptom of NEC or between hours 7-10 following the occurrence or presence of a symptom of NEC or between hours 8-11 following the occurrence or presence of a symptom of NEC, between hours 9-12 following the occurrence or presence of a symptom of NEC, between hours 10-13 following the occurrence or presence of a symptom of NEC, between hours 11-14 following the occurrence or presence of a symptom of NEC, between hours 12-15 following the occurrence or presence of a symptom of NEC, between hours 13-16 following the occurrence or presence of a symptom of NEC, between hours 14-17 following the occurrence or presence of a symptom of NEC, between hours 15-18 following the occurrence or presence of a symptom of NEC, between hours 16-19 following the occurrence or presence of a symptom of NEC, between hours 17-20 following the occurrence or presence of a symptom of NEC, between hours 19-22 following the occurrence or presence of a symptom of NEC, between hours 20-23 following the occurrence or presence of a symptom of NEC, between hours 21-24 following the occurrence or presence of a symptom of NEC, between hours 12-48 following the occurrence or presence of a symptom of NEC, between hours 24-36 following after the occurrence or presence of a symptom of NEC, between hours 36-48 following the occurrence or presence of a symptom of NEC or between hours 48-72 following the occurrence or presence of a symptom of NEC.

The term “within 24 hours after birth” refers to administering at least a first unit dose of stem cell-derived exosomes comprising a HB-EGF product within about 24 hours following birth, and the first dose may be succeeded by subsequent dosing outside the initial 24 hour dosing period.

The term “within 24 hours following the onset of at least one symptom of NEC” refers to administering at least a first unit dose of stem cell-derived exosomes comprising a HB-EGF product within about 24 hours following the first clinical sign or symptom of NEC. The first doses may be succeeded by subsequent dosing outside the initial 24 hour dosing period.

The stem cell-derived exosomes comprising a HB-EGF product may be administered to a patient suffering from an intestinal injury or an infant, including a premature infant, once a day (QD), twice a day (BID), three times a day (TID), four times a day (QID), five times a day (FID), six times a day (HID), seven times a day or 8 times a day. The stem cell-derived exosomes comprising HB-EGF product may be administered alone or in combination with feeding. The stem cell-derived exosomes comprising HB-EGF product may be administered to an infant with formula or breast milk with every feeding or a portion of feedings.

The invention also provides for methods of improving the clinical outcome of a human subject at risk for or suffering from an intestinal injury, such as NEC, or HS- or HS/R- or I/R-induced intestinal injury, comprising administering stem cell-derived exosomes comprising a HB-EGF product in an amount effective to protect the intestine of the human subject from NEC or HS- or HS/R- or I/R-induced intestinal injury.

The methods of the invention may be carried out with any HB-EGF product including recombinant HB-EGF produced in E. coli and HB-EGF produced in yeast. The development of expression systems for the production of recombinant proteins is important for providing a source of protein for research and/or therapeutic use. Expression systems have been developed for both prokaryotic cells such as E. coli, and for eukaryotic cells such as yeast (Saccharomyces, Pichia and Kluyveromyces spp) and mammalian cells.

The invention contemplates treating human subjects of any age including infants, children and adults. The methods of the invention may be carried out in any human subject at risk for or suffering from intestinal injury, such as patients suffering from shock, HS or HS/R. HS may be the result of any type of injury, severe hemorrhaging, trauma, surgery, spontaneous hemorrhaging, or intestinal tissue grafting (transplantation). HS causes hypotension with decreased blood flow to vital organs. Other conditions causing hypotension, although not strictly due to blood loss, may also benefit from treatment with a HB-EGF product, for example, patients with major burns, shock due to sepsis or other causes, and major myocardial infarction to name a few. In certain embodiments, the methods of the invention may be carried out in any human subject other than a subject suffering from necrotizing enterocolitis.

In addition, the invention contemplates treating patients of any age including infants, children and adults suffering from intestinal inflammatory conditions, intestinal infections, Hirschprung's Disease, intestinal dysmotility disorders, intestinal pseudo-obstruction (Ogilvie's Syndrome), inflammatory bowel disease, irritable bowel syndrome or chronic constipation, Crohn's Disease, bowel cancer, colorectal cancers.

### Exosomes

It is known that MSC and NSC protect the intestines from NEC. While SC therapy for injured organs was initially based on the hypothesis that SC engraft in injured tissues and then differentiate into cells that replace damaged cells, engraftment and differentiation may not account for all of their therapeutic effects. Some studies show that less than 1% of transplanted MSC migrate to the injury site, with most trapped in liver, spleen and lungs (Phinney et al. Stem Cells 2007; 25:2896-902). On the other hand, SC may exert their effects via the release of secreted components since treatment response is often greater than can be accounted for by engraftment efficiency (von Bahr et al., Stem Cells 2012; 30:1575-8) and administration of AF-MSC conditioned medium protects the intestines from NEC (Stenson, Gut 2014; 63:218-9). Exosomes are attractive candidates for mediating SC signaling because they are involved in cellular exchange of a complex cargo that is protected from the harsh and degrading conditions of the extracellular environment, and their use may make it possible to treat NEC with a non-cell-based, potentially safer therapy.

Exosomes are small naturally-occurring, secreted, lipid membrane vesicles that carry miRNA, mRNA and proteins, enabling intercellular communication by transfer of these materials between cells. They are formed by invagination of endolysosomal vesicles that form multi-vesicular bodies (MVBs) that are released extracellularly upon fusion with the plasma membrane. They are 40-150 nm in diameter and formed with membranes enriched in cholesterol, sphingomyelin and ceramide. Most contain an evolutionarily conserved set of proteins including tetraspanins (CD81, CD63, CD9), but also contain unique type-specific proteins that reflect their cellular source. Known surface biomarkers for exosomes include CD₉, flotillin and others.

Exosomes have potential as therapeutic agents, vehicles for drug delivery and biomarkers for disease. They have ubiquitous presence in body fluids (breast milk, blood, urine, bile, bronchial fluid) and carry antimicrobial peptides including beta-defensin 2 (Hu et al., PLoS Pathog 2013; 9:e1003261).

In addition, exosomes represent a highly attractive alternative to liposomes as drug delivery vehicles since they avoid the use of toxic or immunogenic synthetic lipids. Similar to liposomes, exosomes have a bilipid membrane and aqueous core, deliver their contents across plasma membranes, and are amenable to loading of therapeutic agents. Their contents can be internalized by other cells as a form of intercellular communication. The presence of genetic material and protein in exosomes implies that such biological materials can be loaded into exosomes. Exosomes are widely distributed in body fluids, making their use likely to be well tolerated. They have preferential homing targets depending on their cell source, and may be amenable to membrane modifications that enhance cell targeting. Systemic administration of modified exosomes has resulted in their targeting of neurons, microglia, and oligodendrocytes in brain (Alvarez-Erviti et al., Nat Biotechnol 2011; 29:341-5). Therapeutic agents can be loaded into exosomes either (i) during their biogenesis by transfection of DNA or mRNA encoding the protein of interest into the cells, and subsequent harvesting of exosomes enriched in the relevant transcript and/or protein in the conditioned medium; or (ii) after isolation by loading small molecules into purified exosomes using transient physical (electroporation) or chemical (lipofection) disruption of the exosome membrane.

### EGF Receptor Agonists

The Epidermal Growth Factor Receptor (EGFR) is a transmembrane glycoprotein that is a member of the protein kinase superfamily. The EGFR is a receptor for members of the epidermal growth factor family. Binding of the protein to a receptor agonist induces receptor dimerization and tyrosine autophosphorylation, and leads to cell proliferation and various other cellular effects (e.g. chemotaxis, cell migration).

The amino acid sequence of the EGF receptor is set out as SEQ ID NO: 16 (Genbank Accession No. NP_005219). EGF receptors are encoded by the nucleotide sequence set out as SEQ ID NO: 15 (Genbank Accession No. NM_005228). The EGF receptor is also known in the art as EGFR, ERBB, HER1, mENA, and PIG61. An EGF receptor agonist is a molecule that binds to and activates the EGF receptor so that the EGF receptor dimerizes with the appropriate partner and induces cellular signaling and ultimately results in an EGF receptor-induced biological effect, such as cell proliferation, cell migration or chemotaxis. Exemplary EGF receptor agonists include epidermal growth factor (EGF), heparin binding EGF (HB-EGF), transforming growth factor-α (TGF-α), amphiregulin, betacellulin, epiregulin, and epigen.

### Epidermal Growth Factor

Epidermal Growth Factor (EGF), also known as beta-urogastrone, URG and HOMG4, is a potent mitogenic and differentiation factor. The amino acid sequence of EGF is set out as SEQ ID NO: 4 (Genbank Accession No. NP_001954). EGF is encoded by the nucleotide sequence set out as SEQ ID NO: 3 (Genbank Accession No. NM_001963).

As used herein, “EGF product” includes EGF proteins comprising about amino acid 1 to about amino acid 1207 of SEQ ID NO: 4; EGF proteins comprising about amino acid 1 to about amino acid 53 of SEQ ID NO: 4; fusion proteins comprising the foregoing EGF proteins; and the foregoing EGF proteins including conservative amino acid substitutions. In a specific embodiment, the EGF product is human EGF(1-53), which is a soluble active polypeptide. Conservative amino acid substitutions are understood by those skilled in the art. The EGF products may be isolated from natural sources, chemically synthesized, or produced by recombinant techniques. In order to obtain EGF products of the invention, EGF precursor proteins may be proteolytically processed in situ. The EGF products may be post-translationally modified depending on the cell chosen as a source for the products.

The EGF products of the invention are contemplated to exhibit one or more biological activities of EGF, such as those described in the experimental data provided herein or any other EGF biological activity known in the art. For example, the EGF products of the invention may exhibit one or more of the following biological activities: cellular mitogenicity in a number of cell types including epithelial cells and smooth muscle cells, cellular survival, cellular migration, cellular differentiation, organ morphogenesis, epithelial cytoprotection, tissue tropism, cardiac function, wound healing, epithelial regeneration, promotion of hormone secretion such as prolactin and human gonadotrophin, pituitary hormones and steroids, and influence glucose metabolism.

The present invention provides for the EGF products encoded by the nucleic acid sequence of SEQ ID NO: 4 or fragments thereof including nucleic acid sequences that hybridize under stringent conditions to the complement of the nucleotides sequence of SEQ ID NO: 3, a polynucleotide which is an allelic variant of SEQ ID NO: 3; or a polynucleotide which encodes a species homolog of SEQ ID NO: 4.

### HB-EGF Polypeptide

The cloning of a cDNA encoding human HB-EGF (or HB-EHM) is described in Higashiyama et al., Science, 251: 936-939 (1991) and in a corresponding international patent application published under the Patent Cooperation Treaty as International Publication No. WO 92/06705 on Apr. 30, 1992. Both publications are hereby incorporated by reference herein in their entirety. In addition, uses of human HB-EGF are taught in U.S. Pat. No. 6,191,109 and International Publication No. WO 2008/134635(Intl. Appl. No. PCT/US08/61772), also incorporated by reference in its entirety.

The sequence of the protein coding portion of the cDNA is set out in SEQ ID NO: 1 herein, while the deduced amino acid sequence is set out in SEQ ID NO: 2. Mature HB-EGF is a secreted protein that is processed from a transmembrane precursor molecule (pro-HB-EGF) via extracellular cleavage. The predicted amino acid sequence of the full length HB-EGF precursor represents a 208 amino acid protein. A span of hydrophobic residues following the translation-initiating methionine is consistent with a secretion signal sequence. Two threonine residues (Thr75 and Thr85 in the precursor protein) are sites for O-glycosylation. Mature HB-EGF consists of at least 86 amino acids (which span residues 63-148 of the precursor molecule), and several microheterogeneous forms of HB-EGF, differing by truncations of 10, 11, 14 and 19 amino acids at the N-terminus have been identified. HB-EGF contains a C-terminal EGF-like domain (amino acid residues 30 to 86 of the mature protein) in which the six cysteine residues characteristic of the EGF family members are conserved and which is probably involved in receptor binding. HB-EGF has an N-terminal extension (amino acid residues 1 to 29 of the mature protein) containing a highly hydrophilic stretch of amino acids to which much of its ability to bind heparin is attributed. Besner et al., Growth Factors, 7: 289-296 (1992), which is hereby incorporated by reference herein, identifies residues 20 to 25 and 36 to 41 of the mature HB-EGF protein as involved in binding cell surface heparin sulfate and indicates that such binding mediates interaction of HB-EGF with the EGF receptor.

As used herein, “HB-EGF product” includes HB-EGF proteins comprising about amino acid 63 to about amino acid 148 of SEQ ID NO: 2 (HB-EGF(63-148)); HB-EGF proteins comprising about amino acid 73 to about amino acid 148 of SEQ ID NO: 2 (HB-EGF(73-148)); HB-EGF proteins comprising about amino acid 74 to about amino acid 148 of SEQ ID NO: 2 (HB-EGF(74-148)); HB-EGF proteins comprising about amino acid 77 to about amino acid 148 of SEQ ID NO: 2 (HB-EGF(77-148)); HB-EGF proteins comprising about amino acid 82 to about amino acid 148 of SEQ ID NO: 2 (HB-EGF(82-148)); HB-EGF proteins comprising a continuous series of amino acids of SEQ ID NO: 2 which exhibit less than 50% homology to EGF and exhibit HB-EGF biological activity, such as those described herein; fusion proteins comprising the foregoing HB-EGF proteins; and the foregoing HB-EGF proteins including conservative amino acid substitutions. In a specific embodiment, the HB-EGF product is human HB-EGF(74-148). Conservative amino acid substitutions are understood by those skilled in the art. The HB-EGF products may be isolated from natural sources known in the art (e.g., the U-937 cell line (ATCC CRL 1593)), chemically synthesized, or produced by recombinant techniques such as disclosed in WO92/06705, supra, the disclosure of which is hereby incorporated by reference. In order to obtain HB-EGF products of the invention, HB-EGF precursor proteins may be proteolytically processed in situ. The HB-EGF products may be post-translationally modified depending on the cell chosen as a source for the products.

The HB-EGF products of the invention are contemplated to exhibit one or more biological activities of HB-EGF, such as those described in the experimental data provided herein or any other HB-EGF biological activity known in the art. One such biological activity is that HB-EGF products compete with HB-EGF for binding to the ErbB-1 receptor and has ErbB-1 agonist activity. In addition, the HB-EGF products of the invention may exhibit one or more of the following biological activities: cellular mitogenicity, cellular chemoattractant, endothelial cell migration, acts as a pro-survival factor (protects against apoptosis), decrease inducible nitric oxide synthase (iNOS) and nitric oxide (NO) production in epithelial cells, decrease nuclear factor-κB (NF-κB) activation, increase eNOS (endothelial nitric oxide synthase) and NO production in endothelial cells, stimulate angiogenesis and promote vasodilatation.

The present invention provides for the HB-EGF products encoded by the nucleic acid sequence of SEQ ID NO: 1 or fragments thereof including nucleic acid sequences that hybridize under stringent conditions to the complement of the nucleotides sequence of SEQ ID NO: 1, a polynucleotide which is an allelic variant of any SEQ ID NO: 1; or a polynucleotide which encodes a species homolog of SEQ ID NO: 2.

### Additional EGF Receptor Agonists

Additional EGF receptor agonists include: Transforming Growth Factor-α (TGF-α), also known as TFGA, which has the amino acid sequence set out as SEQ ID NO: 6 (Genbank Accession No. NP_001093161), and is encoded by the nucleotide sequence set out as SEQ ID NO: 5 (Genbank Accession No. NM_001099691); amphiregulin, also known as AR, SDGF, CRDGF, and MGC13647, which has the amino acid sequence set out as SEQ ID NO: 8 (Genbank Accession No. NP_001648), and is encoded by the nucleotide sequence set out as SEQ ID NO: 7 (Genbank Accession No. NM_001657); betacellulin (BTG) which has the amino acid sequence set out as SEQ ID NO: 10 (Genbank Accession No. NP_001720), and is encoded by the nucleotide sequence set out as SEQ ID NO: 9 (Genbank Accession No. NM_001729); Epiregulin (EREG), also known as ER, which has the amino acid sequence set out as SEQ ID NO: 12 (Genbank Accession No. NP_001423) and is encoded by the nucleotide sequence set out as SEQ ID NO: 11 (Genbank Accession No. NM_001432); and epigen (EPGN) also known as epithelial mitogen homolog, EPG, PRO9904, ALGV3072, FLJ75542, which has the amino acid sequence set out as SEQ ID NO: 14 (Genbank Accession No. NP_001013460), and is encoded by the nucleotide sequence set out as SEQ ID NO: 13 (Genbank Accession No. NM_001013442).

The EGF receptor agonists also may be encoded by nucleotide sequences that are substantially equivalent to any of the EGF receptor agonists polynucleotides recited above. Polynucleotides according to the invention can have at least, e.g., 65%, 70%, 75%, 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the polynucleotides recited above. Preferred computer program methods to determine identity and similarity between two sequences include, but are not limited to, the GCG program package, including GAP (Devereux et al., Nucl. Acid. Res., 12: 387, 1984; Genetics Computer Group, University of Wisconsin, Madison, Wis.), BLASTP, BLASTN, and FASTA (Altschul et al., J. Mol. Biol., 215: 403-410, 1990). The BLASTX program is publicly available from the National Center for Biotechnology Information (NCBI) and other sources (BLAST Manual, Altschul et al. NCB/NLM/NIH Bethesda, Md. 20894; Altschul et al., J. Mol. Biol., 215: 403-410, 1990). The well known Smith Waterman algorithm may also be used to determine identity.

Included within the scope of the nucleic acid sequences of the invention are nucleic acid sequence fragments that hybridize under stringent conditions to any of SEQ ID NOS: 1, 3, 5, 7, 9, 11 and 13, or compliments thereof, which fragment is greater than about 5 nucleotides, preferably 7 nucleotides, more preferably greater than 9 nucleotides and most preferably greater than 17 nucleotides. Fragments of, e.g., 15, 17, or 20 nucleotides or more that are selective for (i.e., specifically hybridize to any one of the polynucleotides of the invention) are contemplated.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The EGF receptor agonists of the invention include, but are not limited to, a polypeptide comprising: the amino acid sequences encoded by the nucleotide sequence of any one of SEQ ID NOS: 1, 3, 5, 7, 9, 11 and 13, or the corresponding full length or mature protein. In one embodiment, polypeptides of the invention also include polypeptides preferably with EGF receptor agonist biological activity described herein that are encoded by: (a) an open reading frame contained within any one of the nucleotide sequences set forth as SEQ ID NO: 1, 3, 5, 7, 9, 11 and 13, preferably the open reading frames therein or (b) polynucleotides that hybridize to the complement of the polynucleotides of (a) under stringent hybridization conditions. In another embodiment, polypeptides of the invention also include polypeptides preferably with EGF receptor agonist biological activity described herein that are encoded by: (a) an open reading frame contained within the nucleotide sequences set forth any as SEQ ID NO: 1, 3, 5, 7, 9, 11 and 13, preferably the open reading frames therein or (b) polynucleotides that hybridize to the complement of the polynucleotides of (a) under stringent hybridization conditions.

The EGF receptor agonists of the invention also include biologically active variants of any of the amino acid sequences of SEQ ID NO: 2, 4, 6, 8, 10, 12 and 14; and “substantial equivalents” thereof with at least, e.g., about 65%, about 70%, about 75%, about 80%, about 85%, 86%, 87%, 88%, 89%, at least about 90%, 91%, 92%, 93%, 94%, typically at least about 95%, 96%, 97%, more typically at least about 98%, or most typically at least about 99% amino acid identity) that retain EGF receptor agonist biological activity. Polypeptides encoded by allelic variants may have a similar, increased, or decreased activity compared to polypeptides having the amino acid sequence of any of SEQ ID NO: 2, 4, 6, 8, 10, 12 and 14.

The EGF receptor agonists of the invention include polypeptides with one or more conservative amino acid substitutions that do not affect the biological activity of the polypeptide. Alternatively, the EGF receptor agonist polypeptides of the invention are contemplated to have conservative amino acids substitutions which may or may not alter biological activity. The term “conservative amino acid substitution” refers to a substitution of a native amino acid residue with a nonnative residue, including naturally occurring and nonnaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, according to the methods of “alanine scanning mutagenesis.” Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine.

### Expression of HB-EGF by Stem Cells

The invention provides for transforming or transfecting somatic stem cells, such as MSC or ISC, with a nucleic acid encoding the amino acid sequence of a HB-EGF product. The transformed somatic stem cells are then administered to a patient suffering from an intestinal injury in any of the methods of the invention which results in administration of the HB-EGF product and the somatic stem cell concurrently.

A nucleic acid molecule encoding the amino acid sequence of an HB-EGF product may be inserted into an appropriate expression vector that is functional in stem cells using standard ligation techniques. Exemplary vectors that function in somatic stem cells include bacterial vectors, eukaryotic vectors, plasmids, cosmids, viral vectors, adenovirus vectors and adenovirus associated vectors.

The expression vectors preferably may contain sequences for cloning and expression of exogenous nucleotide sequences. Such sequences may include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

The vector may contain a sequence encoding a “tag”, such as an oligonucleotide molecule located at the 5′ or 3′ end of the HB-EGF product coding sequence; an oligonucleotide sequence encoding polyHis (such as hexaHis), FLAG, hemaglutinin influenza virus (HA) or myc or other tags for which commercially available antibodies exist. This tag may be fused to the HB-EGF product upon expression. A selectable marker gene element encoding a protein necessary for the survival and growth of a host cell grown in a selective culture medium may also be a component of the expression vector. Exemplary selection marker genes include those that encode proteins that complement auxotrophic deficiencies of the cell; or supply critical nutrients not available from complex media.

A leader, or signal, sequence may be used to direct the HB-EGF product out of the stem cell after administration. For example, a nucleotide sequence encoding the signal sequence is positioned in the coding region of the HB-EGF product nucleic acid, or directly at the 5′ end of the HB-EGF coding region. The signal sequence may be homologous or heterologous to the HB-EGF product gene or cDNA, or chemically synthesized. The secretion of the HB-EGF product from the stem cell via the presence of a signal peptide may result in the removal of the signal peptide from the secreted HB-EGF product. The signal sequence may be a component of the vector, or it may be a part of the nucleic acid molecule encoding the HB-EGF product that is inserted into the vector.

The expression vectors used in the methods of the invention may contain a promoter that is recognized by the host organism and operably linked to the nucleic acid sequence encoding the HB-EGF product. Promoters are untranscribed sequences located upstream to the start codon of a structural gene that control the transcription of the structural gene. Inducible promoters initiate increased levels of transcription from DNA under their control in response to some change in culture conditions, such as the presence or absence of a nutrient or a change in temperature. Alternatively, constitutive promoters initiate continual gene product production with little or no control over gene expression. A large number of promoters, recognized by a variety of potential host cells, are well known. A suitable promoter is operably linked to the nucleic acid molecule encoding the HB-EGF product. The native HB-EGF gene promoter sequence may be used to direct amplification and/or expression of a HB-EGF product nucleic acid molecule. A heterologous promoter also may be used to induce greater transcription and higher yields of the HB-EGF product expression as compared to HB-EGF expression induced by the native promoter.

In addition, an enhancer sequence may be inserted into the vector to increase the transcription of a DNA encoding the HB-EGF product. Enhancers are cis-acting elements of DNA, usually about 10-300 bp in length, that act on the promoter to increase transcription. Enhancer sequences available from mammalian genes include globin, elastase, albumin, alpha-feto-protein and insulin. Exemplary viral enhancers that activate eukaryotic promoters include the SV40 enhancer, the cytomegalovirus early promoter enhancer, the polyoma enhancer, and adenovirus enhancers. While an enhancer may be spliced into the vector at a position 5′ or 3′ to a nucleic acid molecule encoding the HB-EGF product, it is typically located at a site 5′ from the promoter.

The transformation of an expression vector encoding a HB-EGF product into a stem cell may be accomplished by well-known methods such as transfection, infection, calcium chloride, electroporation, microinjection, lipofection or the DEAE-dextran method or any other technique known in the art. These methods and other suitable methods are well known in the art, for example, in Sambrook, Molecular Cloning: A Laboratory Manual, Cold Spring Harbor Laboratory Press; 3rd ed., 2001.

### Somatic Stem Cells

Stem cells are cells with the ability to divide for indefinite periods in culture to give rise to specialized cells. The term “somatic stem cell” or “adult stem cell” refers to undifferentiated cells, found among differentiated cells within a tissue or organ, which has the capacity for self-renewal and differentiation. The somatic stem cells can differentiate to yield some or all of the major specialized cell types of the renewable tissue or organ. The primary role of somatic stem cells is to maintain and repair the tissue in which they are found.

Somatic stem cells may be used for transplantation or may be used to generate SC-derived exosomes for use in any of the methods of the invention. Any type of somatic stem cell may be used to generate the SC-derived exosomes of the invention. Exemplary somatic stem cells include hematopoietic stem cells, mesenchymal stem cells, intestinal stem cells, skeletal stem cells, hepatocyte stem cells, neural stem cells, skin stem cells, endothelial stem cells, mammary stem cells, and neural crest stem cells.

### Mesenchymal Stem Cells

“Mesenchymal stem cells” (MSC) are non-hematopoietic, pluripotent, self-renewing progenitor cells with a characteristic spindle-shaped morphology. These cells are derived from immature embryonic connective tissue (mesoderm layer).

MSC have been shown to contribute to the maintenance and regeneration of various connective tissues. (Pittenger et al., Science 1999; 284(5411):143-7) MSC differentiate into a number of cell types, including chondrocytes, bone, fat, cells that support the formation of blood, and fibrous connective tissue.

MSC are mobilized from bone marrow in response to tissue injury to aid in repair after a variety of end organ injury-models including models of myocardial infarction (Kawada et al., Blood 2004; 104:3581-7), spinal cord injury (Koda et al., Neuroreport 2005; 16:1763-7), renal ischemia/reperfusion injury (Togel et al., Am J Physiol Renal Physiol 2005; 289:F31-42) and intestinal radiation injury (Zhang et al., J Biomed Sci 2008; 15:585-94).

Mesenchymal stem cells may be isolated from various tissues including but not limited to bone marrow (denoted as BM-MSC herein), peripheral blood, blood, placenta, and adipose tissue and amniotic fluid (denoted as AF-MCS herein) Exemplary methods of isolating mesenchymal stem cells from bone marrow are described in (Phinney et al., J Cell Biochem 1999; 72(4):570-85), from amniotic fluid (Baghaban et al., Arch Iran Med 2011; 14(2):96-103), from peripheral blood are described by Kassis et al. (Bone Marrow Transplant. 2006 May; 37(10):967-76), from placental tissue are described by Zhang et al. (Chinese Medical Journal, 2004, 117 (6):882-887), from adipose tissue, placental and cord blood mesenchymal stem cells are described by Kern et al. (Stem Cells, 2006; 24:1294-1301).

The mesenchymal stem cells may be characterized using structural phenotypes. For example, the cells of the present invention may show morphology similar to that of mesenchymal stem cells (a spindle-like morphology). Alternatively or additionally, the MSC may be characterized by the expression of one or more surface markers. Exemplary MSC surface markers include but are not limited to CD105+, CD29+, CD44+, CD90+, CD73+, CD105+, CD166+, CD49+, SH(1), SH(2), SH(3), SH(4), CD14−, CD34−, CD45−, CD19−, CD5−, CD20−, CD11B−, FMC7− and HLA class 1 negative. Other mesenchymal stem cell markers include but are not limited to tyrosine hydroxylase, nestin and H-NF.

Examples of cells derived from mesenchymal cells include (1) cells of the cardiovascular system such as endothelial cells or cardiac muscle cells or the precursor cells of the cells of the cardiovascular system, and cells having the properties of these cells; (2) cells of any one of bone, cartilage, tendon and skeletal muscle, the precursor cells of the cells of any one of bone, cartilage, tendon, skeletal muscle and adipose tissue, and the cells having the properties of these cells; (3) neural cells or the precursor cells of neural cells, and the cells having the properties of these cells; (4) endocrine cells or the precursor cells of endocrine cells, and the cells having the properties of these cells; (5) hematopoietic cells or the precursor cells of hematopoietic cells, and the cells having the properties of these cells; and (6) hepatocytes or the precursor cells of hepatocytes, and the cells having the properties of these cells.

Methods of mesenchymal cell culture are well known in the art of cell culturing (see, for example, Friedenstein et al., Exp Hematol 1976 4, 267-74; Dexter et al. J Cell Physiol 1977, 91:335-44; and Greenberger, Nature 1978 275, 7524). For example, mesenchymal cells are derived from a source selected from the group consisting of endothelial cells, cardiac muscle cells, bone cells, cartilage cells, tendon cells, skeletal muscle cells, bone cells, cartilage cells, tendon cells, adipose tissue cells, neural cells, endocrine cells, hematopoietic cells, hematopoietic precursor cells, bone marrow cells, and the precursor cells thereof, hepatocytes, and hepatocyte precursor cells.

The marrow or isolated mesenchymal stem cells can be autologous, allogeneic or from xenogeneic sources, and can be embryonic or from post-natal sources. Bone marrow cells may be obtained from iliac crest, femora, tibiae, spine, rib or other medullary spaces. Other sources of human mesenchymal stem cells include embryonic yolk sac, placenta, umbilical cord, periosteum, fetal and adolescent skin, and peripheral, circulating blood.

### Intestinal Stem Cells

The lining of the intestines is composed of millions of villi and crypts, which form a barrier against bacterial invasion. The intestinal epithelium is the most rapidly proliferating tissue in adult mammals. Intestinal stem cells (ISCs) are responsible for self-renewal of the epithelium, and also represent a reserve pool of cells that can be activated after injury. The estimated number of stem cells is 4-6 per crypt. (Barker et al., Gastroenterology 2007; 133:1755-1760) Stem cells have been proven to be crucial for the recovery and regeneration of several tissues including the intestinal epithelium. (Vaananen et al., Ann Med 2005; 37:469-479). In the past, ISCs were identified at position +4 from the crypt bottom, directly above the Paneth cells. It is now thought that there may be two populations of ISCs, a slowly cycling quiescent reserve population above the Paneth cells (upper stem cell zone, USZ) (the +4 cells), and a more rapidly cycling (every 24 hours) active pool of crypt base columnar (CBC) cells located between the Paneth cells (lower stem cell zone, LSZ). The more active ISCs may maintain homeostatic regenerative capacity of the intestine with the more quiescent ISCs held in reserve. (Scoville et al., Gastroenterology 2008 136: 849-864) Several signaling pathways including the Wnt/b-catenin, BMP, RTK/PI3K and Notch cascades are critical to ISC self-renewal and proliferation. Among them, Wnt/b-catenin is the signature/signaling pathway, and its downstream regulated genes represent potential ISC markers. The Wnt/b-catenin target gene LGR5 has been recently identified as a marker for CBC ISCs. (Sato et al., Nature 2009; 459:262-265) Prominin-1 is also expressed in ISC. (Snippert et al., Gastroenterology 2009; 136:2187-2194, Zhu et al., Nature 2009; 457: 603-607).

The integrity of the intestinal epithelium is ensured by pluripotent, self-renewing and proliferative stem cells. Barker et al., Gastroenterology 2007; 133:1755-1760, Potten et al., Cell Prolif 2009; 42:731-750. These cells have only recently been identified using special markers such as Leucine-rich repeat-containing G-protein coupled receptor 5 (LGRS) and prominin-1/CD133, in addition to classic+4 long retention cell characteristics. Barker et al., Nature 2007; 449:1003-1007, Snippert et al., Gastroenterology 2009; 136:2187-2194. Between 4 and 6 stem cells at each crypt base generate epithelial progenitor cells in the transit-amplifying (TA) zone, which subsequently differentiate and maintain intestinal homeostasis. Barker et al., Gastroenterology 2007; 133:1755-1760, Potten et al., Cell Prolif 2009; 42:731-750. They provide a fast-paced renewal of the four differentiated epithelial cell lineages, each of which has distinct important physiologic functions: enterocytes that absorb nutrients, goblet cells that produce protective mucus, Paneth cells that secrete antibacterial proteins and neuroendocrine cells that produce hormones. Scoville et al., Gastroenterology 2008; 134:849-864. Stresses such as intestinal ischemia can harm the intestinal epithelial cell (IEC) lineages, particularly the stem cells, thereby disrupting normal homeostasis and gut barrier function. Stem cells in some organs, including the intestines, have been shown to respond to stress and to promote recovery from injury. Markel et al., J Pediatr Surg 2008; 43:1953-1963. A previous study showed that bone marrow-derived progenitor cells have the ability to regenerate the intestine after injury. Gupta et al., Biomacromolecules 2006; 7:2701-2709. However, the role of intestinal stem cells (ISCs) in recovery from NEC is unknown. The ability to protect ISCs in the face of stress may represent a critical step in the prevention and treatment of NEC.

Cell surface markers for ISC include but are not limited to LGRS and prominin-1 (Barker et al., Nature 2007; 449:1003-1007, Snippert et al., Gastroenterology 2009; 136:2187-2194, Lee et al., Nat Neurosci 2005; 8:723-729, Zhu et al., Nature 2009; 457: 603-607, Chen et al., Growth Factors 2010; 28:82-97).

### Embryonic Stem Cells

The methods of the invention may be carried out with SC-derived exosomes that were generated by embryonic stem cells. Embryonic stem cells (ESC) are derived from embryos that were developed from eggs that have been fertilized using in vitro fertilization. Procedures for isolating and growing human primordial stem cells are described in U.S. Pat. No. 6,090,622. Human embryonic stem cells (hESCs) can be isolated from human blastocysts obtained from human in vivo preimplantation embryos, in vitro fertilized embryos, or one-cell human embryos expanded to the blastocyst stage (Bongso et al., Hum. Reprod. 4:706, 1989). Human embryos can be cultured to the blastocyst stage in G1.2 and G2.2 medium (Gardner et al., Fertil. Steril. 69:84, 1998). The zona pellucida is removed from blastocysts by brief exposure to pronase. The inner cell masses can be isolated by immunosurgery or by mechanical separation, and are plated on mouse embryonic feeder layers, or in an appropriate culture system. Inner cell mass-derived outgrowths are then dissociated into clumps using calcium and magnesium-free phosphate-buffered saline (PBS) with 1 mM EDTA, using dispase, collagenase, or trypsin, or by mechanical dissociation with a micropipette. The dissociated cells are then replated for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli.

The ESC may be cultured under conditions that support the substantially undifferentiation growth of the primordial stem cells using any suitable cell culture technique known in the art. For example, the ESCs may be grown on synthetic or purified extracellular matrix using methods standard in the art. Alternatively, the ESC may be grown on extracellular matrix that contains laminin or a growth-arrested murine or human feeder cell layer (e.g., a human foreskin fibroblast cell layer) and maintained in a serum-free growth environment.

Cell surface markers for ESC include, but are not limited to, alkine phosphatase, CD30, Cripto (TDGF-1), GCTM-2, Genesis, Germ cell nuclear factor, OCT-4/POU5F1, SSEA-3, SSEA-4, stem cell factor (SCF or c-kit ligand), TRA-1-60 and TRA-1-81.

### Stem Cell Administration

The methods of the invention may further comprise administering isolated somatic stem cells, such as MSC or ISC in combination with administration of the SC-derived exosomes or instead of administration of the SC-derived exosomes. The term “isolated” refers to a cell that has been removed from its in vivo location (e.g. bone marrow, neural tissue). Preferably the isolated cell is substantially free from other substances (e.g., other cell types) that are present in its in vivo location. The stem cells of the present invention may be isolated or obtained using any technique, preferably known to those skilled in the art.

The somatic stem cells used in any of the methods of the invention may be obtained from any autologous or non-autologous (i.e., allogeneic or xenogeneic) human donor. For example, cells may be isolated from a donor subject. The somatic stem cells of the present invention may be administered to the treated subject using a variety of transplantation approaches, depending on the site of implantation.

Methods of culturing stem cells ex vivo are well known in the art. For example, see “Culture of Animal Cells—A Manual of Basic Technique” by Freshney, Wiley-Liss, N.Y. (1994), Third Edition, the teachings of which are hereby incorporated by reference.

Culture medium compositions typically include essential amino acids, salts, vitamins, minerals, trace metals, sugars, lipids and nucleosides. Cell culture medium supplies the necessary components to meet the nutritional needs for cells to grow in a controlled, artificial and in vitro environment. Nutrient formulations, pH, and osmolarity vary in accordance with parameters such as cell type, cell density, and the culture system employed. Many cell culture medium formulations are known in the art and a number of media are commercially available.

Once the culture medium is incubated with cells, it is known to those skilled in the art as “conditioned medium”. Conditioned medium contains many of the original components of the medium, as well as a variety of cellular metabolites and secreted proteins, including, for example, biologically active growth factors, inflammatory mediators and other extracellular proteins.

Preconditioned media ingredients include, but are not limited to those described below. Additionally, the concentration of the ingredients is well known to one of ordinary skill in the art. See, for example, Methods For Preparation Of Media, Supplements and Substrate for Serum-free Animal Cell Cultures. The ingredients include amino-acids (both D and/or L-amino acids) such as glutamine, alanine, arginine, asparagine, cystine, glutamic acid, glycine, histidine, isoleucine, leucine, lysine, methionine, phenylalanine, proline, serine, threonine, tryptophan, tyrosine, and vatine and their derivatives; acid soluble subgroups such as thiamine, ascorbic acid, ferric compounds, ferrous compounds, purines, glutathione and monobasic sodium phosphates.

Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers.

Other ingredients often used in media formulations include fat soluble vitamins (including A, D, E and K) steroids and their derivatives, cholesterol, fatty acids and lipids Tween 80, 2-mercaptoethanol pyramidines as well as a variety of supplements including serum (fetal, horse, calf, etc.), proteins (insulin, transferrin, growth factors, hormones, etc.) antibiotics (gentamicin, penicillin, streptomycin, amphotericin B, etc.) whole egg ultra filtrate, and attachment factors (fibronectins, vitronectins, collagens, laminins, tenascins, etc.). The media may or may not need to be supplemented with growth factors and other proteins such as attachment factors.

The term “transplantation,” “cell replacement” or “grafting” are used interchangeably herein and refer to the introduction of the somatic stem cells of the present invention to target tissue such as areas of intestinal injury. The cells can be derived from the transplantation recipient or from an allogeneic or xenogeneic donor.

For example, the cells can be grafted into the intestine. Conditions for successful transplantation include: (i) viability of the implant; (ii) retention of the graft at the site of transplantation; and (iii) minimum amount of pathological reaction at the site of transplantation.

For administration of the stem cells, an effective amount of the stem cells are diluted in suitable carriers. Exemplary carriers include phosphate buffered saline (PBS), culture medium and other buffered solutions.

The isolated stem cells may be administered by intravenous injection, by intraperitoneal injection or by preparing a cavity by surgical means to expose the intestine and then depositing the graft into the cavity. The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to the injured region. Then following transplantation, the cells preferably migrate to the damaged area.

Since non-autologous stems cell may induce an immune reaction when administered to the body, steps may be necessary to decrease the likelihood of rejection of the stem cells. These steps include suppressing the recipient immune system or encapsulating the non-autologous stem cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag et al. Adv Drug Deliv Rev. 2000; 42: 29-64). Exemplary methods of preparing microcapsules include those made of alginate and alpha-phenoxycinnamylidene-acetylated poly(allylamine) (Lu et al., Biotechnol Bioeng. 2000, 70: 479-83) and photosensitive poly(allylamine alpha-cyanocinnamylideneacetate) (J Microencapsul. 2000, 17: 245-51). In addition, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia. et al. Biomaterials. 2002 23: 849-56).

Other microcapsules are based on alginate, a marine polysaccharide (Sambanis et al., Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple et al., J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams Med Device Technol. 1999, 10: 6-9; Desai, Expert Opin Biol Ther. 2002, 2: 633-46).

Examples of immunosuppressive agents that may be administered in conjunction with the methods of the invention include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNFα. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

### Necrotizing Enterocolitis

Necrotizing enterocolitis (NEC) is the most common gastrointestinal emergency in premature newborn infants (Schnabl et al., World J Gastroenterol 14:2142-2161, 2008; Kliegman et al., N Engl J Med 310:1093-103, 1984). With aggressive management leading to the salvage of premature infants from the pulmonary standpoint, the incidence of NEC is increasing, and it is thought that NEC will soon replace pulmonary insufficiency as the leading cause of death in premature infants (Lee et al., Semin Neonatol 8:449-59, 2003). The mortality of this disease ranges from 20% to 50%, resulting in over 1000 infant deaths in this country each year (Caplan et al., Pediatr 13: 111-115, 2001). Like other diseases manifested by severe intestinal injury, NEC can cause the dysregulated inflammation characteristic of the systemic inflammatory response syndrome (SIRS), potentially resulting in multiple organ dysfunction syndrome (MODS) and death. Evidence suggests that the risk factors for NEC, namely formula feeding, intestinal ischemia and bacterial colonization, stimulate proinflammatory mediators that in turn activate a series of events culminating in necrosis of the bowel (Caplan et al., Pediatr 13: 111-115, 2001). Survivors of acute NEC frequently develop malabsorption, malnutrition, total parenteral nutrition-related complications, intestinal strictures and short bowel syndrome (Caplan et al., Pediatr 13:111-115, 2001).

Since prematurity is the single most important risk factor for NEC, it is possible that absent or reduced levels of specific factors that are normally expressed during later periods of gestation may contribute to the development of this condition. With this in mind, exogenous replacement of key factors may be clinically valuable as a means to reduce the incidence of NEC. Several potential preventive strategies have aimed at induction of gastrointestinal maturation with steroids, improvement in host defense with breast milk feeding or oral immunoglobulins, change in bacterial colonization with antibiotics, probiotics or feeding modifications, and reduction or antagonism of inflammatory mediators, none of which have led to consistently positive therapeutic results (Feng et al., Semin Pediatr Surg 14:167-74, 2005).

### Hemorrhagic Shock

Shock is a state of inadequate perfusion, which does not sustain the physiologic needs of organ tissues. Hemorrhagic shock (HS) refers to shock that is caused by blood loss that exceeds the ability of the body to compensate and to provide adequate tissue perfusion and oxygenation. HS is frequently caused by trauma, but also may be caused by spontaneous hemorrhage (e.g., GI bleeding, childbirth), surgery, and other causes. Frequently, an acute bleeding episode will cause HS, but HS may also occur in chronic conditions with subacute blood loss.

Untreated HS can lead to death. Without intervention, a classic trimodal distribution is seen in severe HS. An initial peak of mortality occurs within minutes of hemorrhage due to immediate exsanguination. Another peak occurs after 1 to several hours due to progressive decompensation. A third peak occurs days to weeks ater due to sepsis and organ failure. Therefore, the methods of the invention preferably are carried out during the early stages of HS such as after or during the initial peak, or before or during the second peak (1 to several hours after the initial hemorrhage).

A person in shock has extremely low blood pressure. Depending on the specific cause and type of shock, symptoms will include one or more of the following: anxiety, agitation, confusion, pale, cool and clammy skin, low or no urine production, bluish lips and fingernails, dizziness, light-headedness, faintness, profuse sweating, rapid but weak pulse, shallow breathing, chest pain and unconsciousness.

Resuscitation during or after HS/R is known to have deleterious effects on the blood vessels of the patient. For example, HS/R is characterized by progressive deterioration of mesenteric blood flow. In addition, progressive intestinal hypoperfusion after HS/R contributes to loss of the gut mucosal barrier and to hypoxia-induced intestinal inflammation, both of which are critical to the initiation of MODS after HS/R.

### The Role of HB-EGF in Intestinal Cytoprotection

Induction and activation of the EGF receptor have been demonstrated in different tissues, including the intestines, during hypoxia and after ischemia. (Ellis et al., Biochem. J. 354:99-106, 2001; Lin et al., J Lab Clin Med; 125:724-33, 1995; Nishi et al., Cancer Res 62:827-34, 2002; Sondeen et al., J Lab Clin Med 134:641-8, 1999; Yano et al., Nephron 81:230-3, 1999). Previous studies have shown that HB-EGF mRNA and protein are induced after exposure of intestinal epithelial cells to anoxia/reoxygenation (A/R) in vitro, and after intestinal I/R injury in vivo. (Xia et al., J Invest Surg 16:57-63, 2003). Hypoxia and I/R have been found to induce HB-EGF transcription and protein synthesis in different tissues including the brain and kidney. (Homma et al., J Clin Invest 96:1018-25, 1995; Jin et al., J Neurosci 22:5365-73, 2002; Kawahara et al., J Cereb Blood Flow Metab 19:307-20, 1999; Sakai et al., J. Clin. Invest.; 99:2128-2138, 1997). During the early phases of hypoxia and oxidative stress, activation of EGFR and shedding of proHB-EGF occur, leading to immediate availability of soluble HB-EGF protein for targeting via autocrine or paracrine pathways. HB-EGF shedding is followed by the induction of transcription and de novo synthesis of HB-EGF (El-Assal et al., Semin Pediatr Surg 13:2-10, 2004).

Intestinal epithelium undergoes a dynamic and continuous process of renewal and replacement with a turnover time of 3-6 days. (Potten et al., Am J Physiol 273:G253-7, 1997). Depending more on the depth of injury rather than the total surface area affected, the process of healing starts as early as a few minutes after injury (Ikeda et al., Dig Dis Sci 2002; 47:590-601, 2002). The most important priority during intestinal regeneration is reconstitution of epithelial cell continuity, allowing restoration of barrier function and prevention of systemic toxic complications. This is achieved by rapid epithelial cell migration from the wound edge, a process known as “restitution” (Ikeda et al., Dig Dis Sci 47:590-601, 2002; McCormack et al., Am J Physiol; 263:G426-35, 1992; Moore et al. Am J Physiol 257:G274-83, 1989; Moore et al. Gastroenterology 102:119-30, 1992). Early migration of goblet cells, which are more resistant to ischemia-induced cell death than enterocytes, serves as a source of both cell lining and mucous secretion, thus promoting rapid recovery of intestinal barrier function (Ikeda et al., Dig Dis Sci 47:590-601, 2002). Complete intestinal repair is achieved by proliferation and differentiation of crypt epithelium, which does not occur as early as restitution. Following administration of HB-EGF to rats exposed to intestinal I/R, a significant improvement in intestinal healing characterized by reduced mucosal damage was observed (Pillai et al., J Surg Res 87:225-31, 1999). In the early phase of intestinal healing HB-EGF was shown to induce intestinal restitution, (El-Assal et al., Gastroenterology 129:609-25, 2005) whereas in the later phase of healing HB-EGF promotes crypt cell proliferation (Xia et al., J Pediatr Surg 37:1081-7; 2002). In addition, the effects of HB-EGF in inducing restitution are mediated by both the PI3-kinase and MAPK intracellular signaling pathways (El-Assal et al. Gastroenterology 129:609-25, 2005). HB-EGF administration leads to preservation of gut barrier function and intestinal permeability after intestinal I/R (El-Assal et al. Gastroenterology 129:609-25, 2005), with resultant decrease in bacterial translocation (Xia et al., J Pediatr Surg 37:1081-7; 2002). It is important to note that the protective effects of HB-EGF administration are seen even when the growth factor is administered during or after the ischemic interval has already occurred (Martin et al., J Pediatr Surg. 40:1741-7, 2005). Thus, prophylactic administration of HB-EGF prior to ischemia is not required. Most importantly, HB-EGF improves survival in rats exposed to intestinal I/R injury (Pillai et al., J Surg Res 87:225-31, 1999).

Additional studies demonstrated that treatment with HB-EGF reduced the generation of ROS in rats exposed to intestinal I/R in vivo and in leukocytes exposed to ROS-inducing stimuli in vitro (Kuhn et al., Antioxid Redox Signal 4:639-46, 2002). HB-EGF also preserved intestinal epithelial cell ATP levels in cells exposed to hypoxia (Pillai et al., J. Pediatr. Surg. 33:973-979, 1998). HB-EGF is known to downregulate expression of adhesion molecules including P- and E-selectin and intercellular adhesion molecule-1 (ICAM-1)/vascular cell adhesion molecule-1 (VCAM-1) after intestinal I/R (Xia et al., J Pediatr Surg 38:434-9. 2003). Downregulation of adhesion molecules was followed by reduced infiltration of leukocytes, which are critical mediators of I/R (Xia et al., J Pediatr Surg 38:434-9. 2003).

Exposure of intestinal epithelium to I/R results in cell death, with apoptosis rather than necrosis as the major mechanism of cell death. One of the unique functions of HB-EGF is its ability to protect against apoptotic cell death. sHB-EGF is known to protect enterocytes from hypoxia-induced intestinal necrosis (Pillai et al., J. Pediatr. Surg. 33:973-979, 1998) and from pro-inflammatory cytokine-induced apoptosis (Michalsky et al., J Pediatr Surg 36:1130-5. 2001) in vitro. HB-EGF is also known to act as a pro-survival factor in cells exposed to various forms of stress including mechanical stress, serum starvation and exposure to cytotoxic agents. Recent studies have demonstrated that HB-EGF decreases intestinal epithelial cell apoptosis in vivo in a rat model of necrotizing enterocolitis (Feng et al., J Pediatr Surg 2006 41(4):742-7)

Nitric oxide (NO) is another mediator of I/R-induced apoptosis and intestinal mucosal damage. Despite the protective effect of constitutive NO, there is ample evidence that high levels of NO induce apoptosis and mediate tissue damage in different cell types including intestinal epithelial cells during I/R. iNOS (inducible nitric oxide synthase) inhibitors led to attenuated NO production and decreased hypoxia-induced intestinal apoptosis with preservation of gut barrier function in rats with endotoxemia. Furthermore, iNOS knock-out mice are more resistant to intestinal I/R-induced mucosal injury. Collectively, these studies clearly indicate that reduction of iNOS can decrease I/R-induced intestinal damage. HB-EGF downregulates cytokine-induced iNOS and NO production in intestinal epithelial cells in vitro, and I/R-induced intestinal iNOS expression and serum NO levels in vivo. HB-EGF has been shown to decrease iNOS and NO production in intestinal epithelial cells, which is dependent upon its ability to decrease nuclear factor-κB (NF-κB) activation in a PI3-kinase dependent fashion. Reduction of I/R-induced overproduction of NO in IEC represents an additional cytoprotective mechanism of HB-EGF.

HB-EGF is a hypoxia- and stress-inducible gene that is involved in reduction of I/R-induced tissue damage. It promotes structural recovery after I/R by enhancing cell proliferation and by inducing migration of healthy epithelial cells from the edge of damaged tissues. In addition to promoting healing based on its positive trophic effects, HB-EGF also protects the intestine by decreasing leukocyte infiltration and production of injurious mediators after injury, thus protecting epithelial cells from apoptosis and necrosis. It is likely that reducing I/R-induced IEC death will ameliorate intestinal damage and reduce systemic complications.

### Pharmaceutical Compositions

The administration of exosomes of the invention and/or a HB-EGF product is preferably accomplished with a pharmaceutical composition comprising a exosomes of the invention and/or a HB-EGF product and a pharmaceutically acceptable carrier. The carrier may be in a wide variety of forms depending on the route of administration. Suitable liquid carriers include saline, PBS, lactated Ringer solution, human plasma, human albumin solution, 5% dextrose and mixtures thereof. The route of administration may be oral, rectal, parenteral, intraluminally, or through a nasogastric or orogastric tube (enteral). Examples of parenteral routes of administration are intravenous, intra-arterial, intraperitoneal, intramuscular or subcutaneous injection or infusion.

A preferred route of administration of the present invention is the enteral route. Therefore, the present invention contemplates that the acid stability of HB-EGF is a unique factor as compared to, for example, EGF. For example, the pharmaceutical composition of the invention may also include other ingredients to aid solubility, or for buffering or preservation purposes. Pharmaceutical compositions containing a HB-EGF product may comprise the HB-EGF product at a concentration of about 100 to 1000 μg/kg in saline. Suitable doses are in the range from 100-140 μg/kg, or 100-110 μg/kg, or 110-120 μg/kg, or 120-130 μg/kg, or 120-140 μg/kg, or 130-140 μg/kg, or 500-700 μg/kg, or 600-800 μg/kg or 800-1000 μg/kg. Preferred doses include 100 μg/kg, 120 μg/kg, 140 μg/kg and 600 μg/kg administered enterally once a day. Additional preferred doses may be administered once, twice, three, four, five, six or seven or eight times a day enterally.

The pharmaceutical compositions of exosomes of the invention and/or a HB-EGF product are administered as methods of the invention include an exosomes or a HB-EGF product which is associated or attached to a carrier that assists in stabilizing the agonist during administration. For example, the invention contemplates administering an exosomes or a HB-EGF product associated with a carrier that prevent digestion in the duodenal fluids such as polymers, phospholipids, hydrogels, polysaccharides and prodrugs, microparticles or nanoparticles. The pharmaceutical compositions may also comprise pH sensitive coatings or carriers for controlled release, pH independent biodegradable coatings or carriers or microbially controlled coatings or carriers.

The dose of exosomes and/or a HB-EGF product may also be administered intravenously. In addition, the dose of exosomes and/or the HB-EGF product may be administered as a bolus, either once at the onset of therapy or at various time points during the course of therapy, such as every four hours, or may be infused for instance at the rate of about 0.01 μg/kg/h to about 5 μg/kg/h during the course of therapy until the patient shows signs of clinical improvement. Addition of other bioactive compounds e.g., antibiotics, free radical scavenging or conversion materials (e.g., vitamin E, beta-carotene, BHT, ascorbic acid, and superoxide dimutase), fibrolynic agents (e.g., plasminogen activators), and slow-release polymers to the HB-EGF product or separate administration of the other bioactive compounds is also contemplated.

As used herein, “pathological conditions associated with intestinal ischemia” includes conditions which directly or indirectly cause intestinal ischemia (e.g., premature birth, birth asphyxia, congenital heart disease, cardiac disease, polycythemia, hypoxia, exchange transfusions, low-flow states, atherosclerosis, embolisms or arterial spasms, ischemia resulting from vessel occlusions in other segments of the bowel, ischemic colitis, and intestinal torsion such as occurs in infants and particularly in animals) and conditions which are directly or indirectly caused by intestinal ischemia (e.g., necrotizing enterocolitis, shock, sepsis, and intestinal angina). Thus, the present invention contemplates administration of an exosomes and/or HB-EGF product to patients in need of such treatment including patients at risk for intestinal ischemia, patients suffering from intestinal ischemia, and patients recovering from intestinal ischemia. The administration of an exosomes and/or HB-EGF product to patients is contemplated in both the pediatric and adult populations.

More particularly, the invention contemplates a method of reducing necrosis associated with intestinal ischemia comprising administering an exosomes and/or HB-EGF product, to a patient at risk for, suffering from, or recovering from intestinal ischemia. Also contemplated is a method of protecting intestinal epithelial cells from hypoxia comprising exposing the cells to a HB-EGF product. Administration of, or exposure to, HB-EGF products reduces lactate dehydrogenase efflux from intestinal epithelial cells, maintains F-actin structure in intestinal epithelial cells, increases ATP levels in intestinal epithelial cells, and induces proliferation of intestinal epithelial cells.

In view of the efficacy of HB-EGF in protecting intestinal tissue from ischemic events, it is contemplated that HB-EGF has a similar protective effect on myocardial, renal, spleen, lung, brain and liver tissue.

### Administration to Pediatric Patients

Intestinal injury related to an ischemic event is a major risk factor for neonatal development of necrotizing enterocolitis (NEC). NEC accounts for approximately 15% of all deaths occurring after one week of life in small premature infants. Although most babies who develop NEC are born prematurely, approximately 10% of babies with NEC are full-term infants. Babies with NEC often suffer severe consequences of the disease ranging from loss of a portion of the intestinal tract to the entire intestinal tract. At present, there are no known therapies to decrease the incidence of NEC in neonates.

Babies considered to be at risk for NEC are those who are premature (less than 36 weeks gestation) or those who are full-term but exhibit, e.g., prenatal asphyxia, shock, sepsis, or congenital heart disease. The presence and severity of NEC is graded using the staging system of Bell et al., J. Ped. Surg., 15:569 (1980) as follows:

| | |
|---|---|
| Stage I | Any one or more historical factors producing perinatal stress |
| (Suspected | Systemic manifestations - temperature instability, lethargy, |
| NEC) | apnea, bradycardia |
| | Gastrointestinal manifestations - poor feeding, increased |
| | pregavage residuals, emesis (may be bilious or test positive |
| | for occult blood), mild abdominal distention, occult blood in |
| | stool (no fissure) |
| Stage II | Any one or more historical factors |
| (Definite | Above signs and symptoms plus persistant occult or gross |
| NEC) | gastrointestinal bleeding, marked abdominal distention |
| | Abdominal radiographs showing significant intestinal |
| | distention with ileus, small-bowel separation (edema in |
| | bowel wall or peritoneal fluid), unchanging or persistent |
| | “rigid” bowel loops, pneumatosis intestinalls, portal venous |
| | gas |
| Stage III | Any one or more historical factors |
| (Advanced | Above signs and symptoms plus deterioration of vital signs, |
| NEC) | evidence of septic shock, or marked gastrointestinal |
| | hemorrhage |
| | Abdominal radiographs showing pneumoperitoneum in |
| | addition to findings listed for Stage II |

Babies at risk for or exhibiting NEC are treated as follows. Patients receive a daily liquid suspension of HB-EGF (e.g. about 1 mg/kg in saline or less). The medications are delivered via a nasogastric or orogastric tube if one is in place, or orally if there is no nasogastric or orogastric tube in place.

### BRIEF DESCRIPTION OF DRAWINGS

FIG. 1 depicts characterization of NSC-derived exosomes (A) Enteric NSC were harvested from intestines of E11.5 mice and expanded as neurospheres. (B) Condition medium was collected and exosomes purified with the PureExo Exosome Isolation Kit and stained with the red fluorescent dye PKH 26. (C) Transmission EM. Scale bar=100 nm. (D) Dynamic light scattering and zeta potential analysis. (E) Western blot shown positive expression for exosome markers CD9 and Flotilin.

FIG. 2 depicts NSC-derived target enteric NSC and injured neurons. Panel (A) shows exosomes stained with PKH26 dye, and applied to a mixed culture of rat intestinal LMMP cells. Red fluorescent dye labeled exosomes were localized only in culture NSCs 24 hours after application. Tuj-1 (mature neuron marker) green; exosomes, red; GFAP (glia cells) green; SMA (smooth muscle cell) green; Nestin (NSCs) green; DAPI (nuclei), LMMP: Longitudinal Muscle-Myenteric Plexus. αSMA: Alpha-smooth muscle actin. Panel (B) shownsPKH26 labeled NSC derived exosomes applied to a mixed culture of rat intestinal LMMP cells 30 min prior to anoxia/Reoxygenation (24 h/24 h) injury. Red cytoplasmic staining in target cells confirms uptake of NSC-derived exosomes in injured intestinal neurons. Panel (C) shows immunohistology specific for cleaved caspase-3 shown decreased neuronal apoptosis in cells transferred with exosomes.

FIG. 3 depicts NSC-derived exosomes can be loaded with HB-EGF and target injured enteric neurons. (a-c) Exosomes were isolated from enteric NSC, stained with PKH26 dye, and applied to a mixed culture of rat intestinal cells 30 min prior to A/R (4 h/24 h) injury. Tuj-1 (mature neuron marker) green; exosomes, red; DAPI (nuclei), blue. Red cytoplasmic staining in target cells confirms uptake of NSC-derived exosomes in injured neurons. (d-g) Enteric NSC were transfected with pGFP-hHB-EGF, exosomes harvested 48 h later were stained with PKH26, and exosomes applied to a mixed culture of rat intestinal cells 30 min prior to A/R (4 h/24 h). Tuj-1 (mature neuron marker) grey; exosomes, red; HB-EGF, green; DAPI (nuclei), blue. Green staining of exosomes confirms HB-EGF loading; yellow merged cytoplasmic staining in target cells confirms that exosomal HB-EGF was transferred to injured neurons.

FIG. 4 depicts NSC-derived exosomes targeting NEC injured intestine and protect against experimental NEC. Panel (A) Pups were subjected to NEC for 24 hours, and then control vehicle or PKH 26-stained exosomes were administered IP. Panel (B) Intestines were removed 48 hours later and examined with Xenon fluorescent imaging. Labeled exosomes (red) localized to injured intestine. Panel (C) Histologic intestinal sections were graded for NEC using the scoring system described before. Panel (D) Representative H&E images demonstrating exosome treatment restores intestinal integrity

FIG. 5 depicts the effect of treatment with mesenchymal stem cells or exosomes has on the severity of NEC.

### DETAILED DESCRIPTION

The following examples illustrate the invention wherein Example 1 describes isolation and characterization of NSC-derived exosomes. Example 2 describes that NSC-derived exosomes target enteric NSC and injured neurons. Example 3 describes a neonatal rat model of experimental necrotizing enterocolitis. Example 4 describes that NSC-derived exosomes target NEC injured intestine and protect against experimental NEC. Example 5 describes that NSC-derived exosomes can be loaded with HB-EGF and target injured enteric neurons in culture. Example 6 describes that SC-derived exosomes can protect the intestines from NEC. Example 7 describes direct transfer of exosomes from donor SC to recipient intestinal/ENS cells protects recipient cells from injury.

### EXAMPLES

### Example 1

Isolation and Characterization of NSC-derived Exosomes

Enteric NSCs were isolated from mid gestational guts of embryonic mice. The harvested cells were immunoselected using magnetic beads conjugated with anti-P75 antibody and cultured in NSC medium. Neurosphere-like bodies were allowed to form and were then passed repeatedly. NSC cultured condition medium were collected and spin down to remove debris.

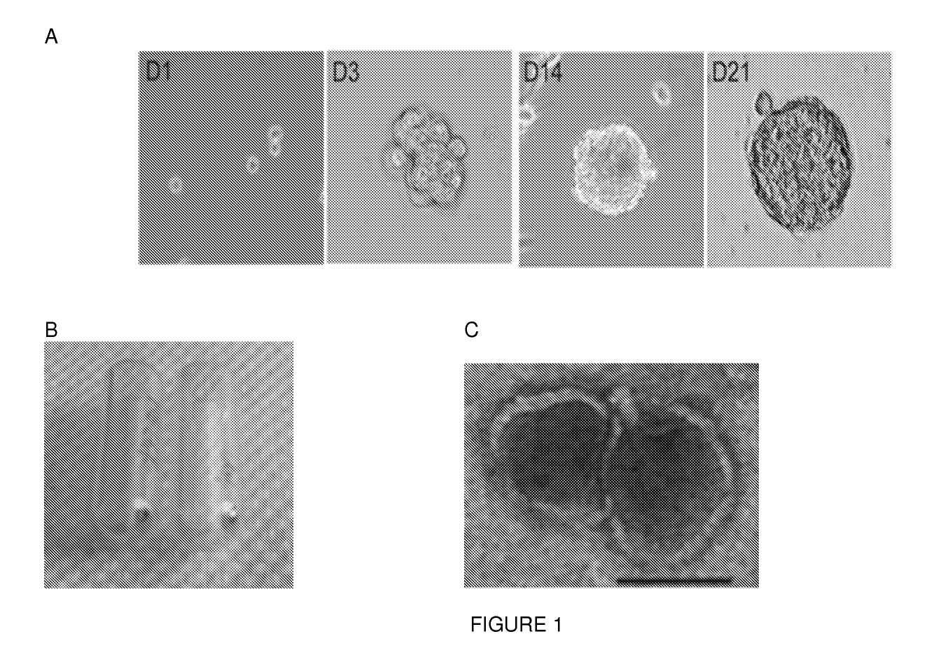

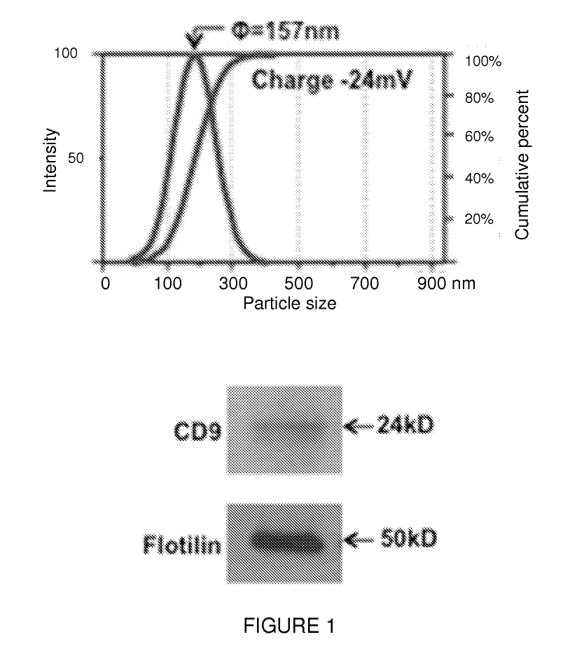

Initially, NSC-derived exosomes were purified from neurosphere conditioned medium (CM) using the PureExo Exosome Isolation kit (101Bio) according to the manufacturer's instructions. Exosomes were then stained with the red fluorescent cell link dye PKH 26 (Sigma) and characterized for size, size distribution, charge, and morphology using dynamic light scattering, zeta potential analysis, and transmission electron microscopy. Electron microscopy (EM) confirmed the presence of 50-150 nm diameter bi-membrane vesicles. Zeta potential analysis revealed a high negative charge of −24 mV. Dynamic light scattering revealed a mean diameter of −157n. Western blot analysis confirmed the presence of the well-characterized marker of murine exosomes tetraspanin CD9, as well as the membrane associated cytoskeletal lipid raft protein flotilin. These results are depicted in FIG. 1.

### Example 2

### NSC-Derived Exosomes Target Enteric NSC and Injured Neurons

The anoxia/reoxgenation cell injured model (Watkins et al., J Surg Res 2012; 177:359-64) was used to demonstrate that the NSC-derived exosomes target enteric NSC and localized to injured neurons. Briefly, intestinal LMMP strips were dissected out from P3-P5 rat pups and were treated with enzymatic digestion to obtain mixed cells which includes: myenteric neurons, glial cell, neural stem cells (NSC), smooth muscular cells. Mixed cells were cultured in DMEM/F12: NeuroBasal medium (v/v=1:1) supplemental with 1×B27 and 10% FBS (Gibco) for 10 days. PKH26 labeled NSC derived exosomes were added to the culture medium of the mixed cells and the targeted cells with exosomes were visualized 24 hours later under fluorescent microcopy. In addition, NSC-derived exosomes were applied to the cultured cells 30 minutes prior to the exposure of the mixed cells to 24 h/24 h anoxia/reoxygenation injury (anoxia chamber was filled with 95% N₂ and 5% CO₂). Cultured cells were then fixed in 4% PFA and exosomes transfer was observed under fluorescent microcopy.

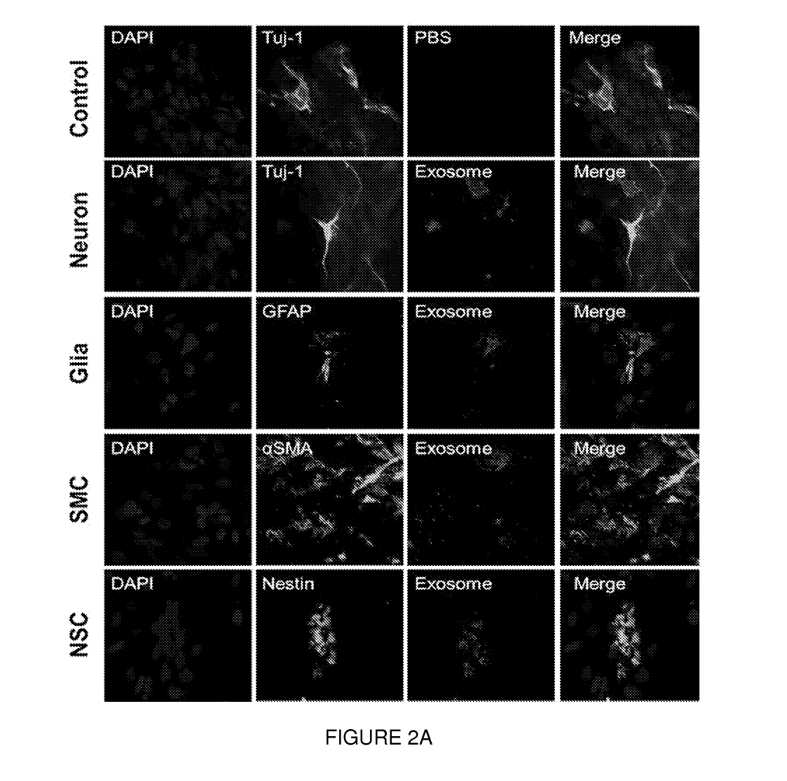

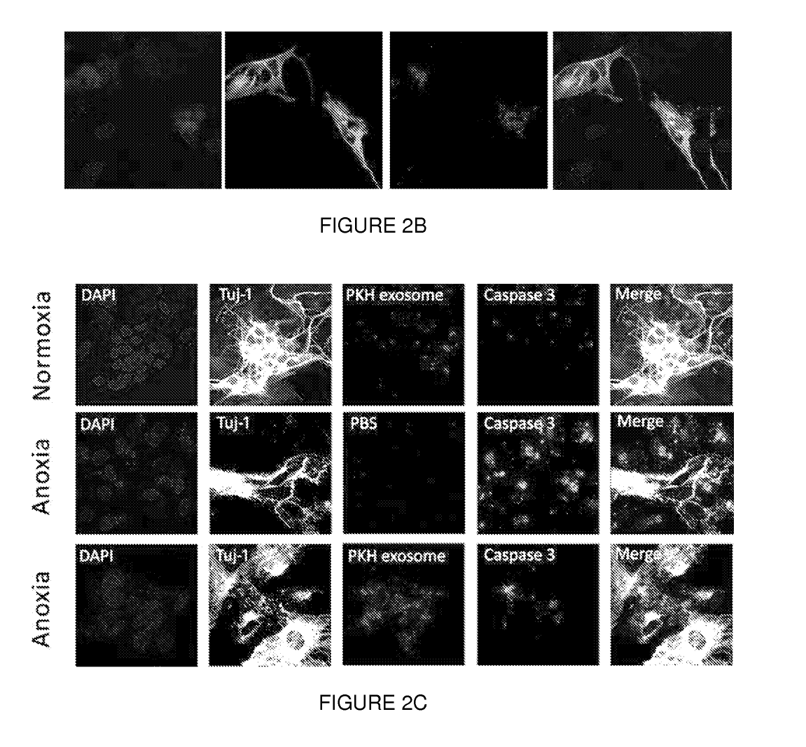

As shown in FIG. 2, NSC-derived exosomes target and protect enteric NSC and injured neurons. The red fluorescent dye labeled exosomes were localized only in culture NSCs 24 hours after application. The PKH26 labeled NSC-derived exosomes were applied to a mixed culture of rat intestinal LMMP cells 30 min prior to anoxia/Reoxygenation (24 h/24 h) injury, and red cytoplasmic staining in target cells confirms uptake of NSC-derived exosomes in injured intestinal neurons. Immunohistochemistry specific for cleaved caspase-3 demonstrated decreased neuronal apoptosis in cells treated with exosomes.

### Example 3

### Neonatal Rat Model of Experimental Necrotizing Enterocolitis

The studies described herein utilize a neonatal rat model of experimental NEC. These experimental protocols were performed according to the guidelines for the ethical treatment of experimental animals and approved by the Institutional Animal Care and Use Committee of Nationwide Children's Hospital (#04203AR). Necrotizing enterocolitis was induced using a modification of the neonatal rat model of NEC initially described by Barlow et al. (J Pediatr Surg 9:587-95, 1974). Pregnant time-dated Sprague-Dawley rats (Harlan Sprague-Dawley, Indianapolis, Ind.) were delivered by C-section under CO2 anesthesia on day 21.5 of gestation. Newborn rats were placed in a neonatal incubator for temperature control. Neonatal rats were fed via gavage with a formula containing 15 g Similac 60/40 (Ross Pediatrics, Columbus, Ohio) in 75 mL Esbilac (Pet-Ag, N.H., IL), a diet that provided 836.8 kJ/kg per day. Feeds were started at 0.1 mL every 4 hours beginning 2 hours after birth and advanced as tolerated up to a maximum of 0.4 mL per feeding by the fourth day of life. Animals were also exposed to a single dose of intragastric lipopolysaccharide (LPS; 2 mg/kg) 8 hours after birth, and were stressed by exposure to hypoxia (100% nitrogen for 1 minute) followed by hypothermia (4° C. for 10 minutes) twice a day beginning immediately after birth and continuing until the end of the experiment. In all experiments, pups were euthanized by cervical dislocation upon the development of any clinical signs of NEC. All remaining animals were sacrificed at the end of experiment at 96 hours after birth.

The HB-EGF used in all experiments was GMP-grade human mature HB-EGF produced in P. pastoris yeast (KBI BioPharma, Inc., Durham, N.C.). EGF was produced in E. coli and purchased from Vybion, Inc. (Ithaca, N.Y.).

To assess the histologic injury score, immediately upon sacrifice, the gastrointestinal tract was carefully removed and visually evaluated for typical signs of NEC including areas of bowel necrosis, intestinal hemorrhage and perforation. Three pieces each of duodenum, jejunum, ileum, and colon from every animal were fixed in 10% formalin for 24 hours, paraffin-embedded, sectioned at 5 μm thickness, and stained with hematoxylin and eosin for histological evaluation of the presence and/or degree of NEC using the NEC histologic injury scoring system described by Caplan et al. (Pediatr Pathol 14:1017-28, 1994). Histological changes in the intestines were graded as follows: grade 0, no damage; grade 1, epithelial cell lifting or separation; grade 2, sloughing of epithelial cells to the mid villus level; grade 3, necrosis of the entire villus; and grade 4, transmural necrosis. All tissues were graded blindly by two independent observers. Tissues with histological scores of 2 or higher were designated as positive for NEC.

Fisher's exact test was used for comparing the incidence of NEC between groups with no adjustments made for multiple comparisons. P-values less then 0.05 were considered statistically significant. All statistical analyses were performed using SAS, (version 9.1,SAS Institute, Cary, N.C.).

### Example 4

### NSC-Derived Exosomes Target NEC Injured Intestine and Protect Against Experimental NEC

Exosomes were stained with the red fluorescent cell linker dye PKH26 to allow their distribution to be tracked. Neonatal mice exposed to experimental NEC for 24 hours as described in Example 3. Newborn rat pups were delivered by C-section and subjected to repeated exposure to hypoxia and hypothermia. These pups were administered subjected to hypertonic feeding for 24 hours and then control vehicle or PKH26-stained exosomes were administered intraperitoneally (IP). Intestines were removed 48 hours later or when NEC clinical signs were observed in these rat pups. The pups were randomly assigned to the following groups: (1) breast feeding only; (2) BF+exosomes IP; (4) NEC; (4) NEC+exosomes IP.

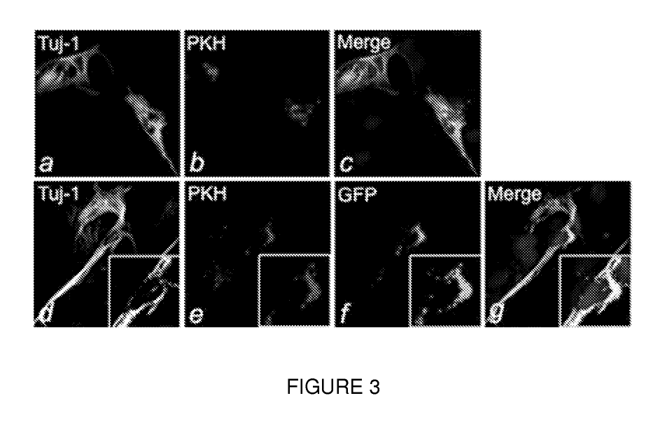

Histologic intestinal sections were graded for NEC using the scoring system. Representative H&E images demonstrating that exosome treatment restores intestinal integrity are shown in FIG. 3.

This experiment demonstrates that NSC-derived exosomes protect intestines from experimental NEC injury. NSC-derived exosomes is a novel non-cell based therapy that protects the ENS from injury during NEC.

### Example 5

### NSC-Derived Exosomes can be Loaded with HB-EGF and Target Injured Enteric Neurons in Culture

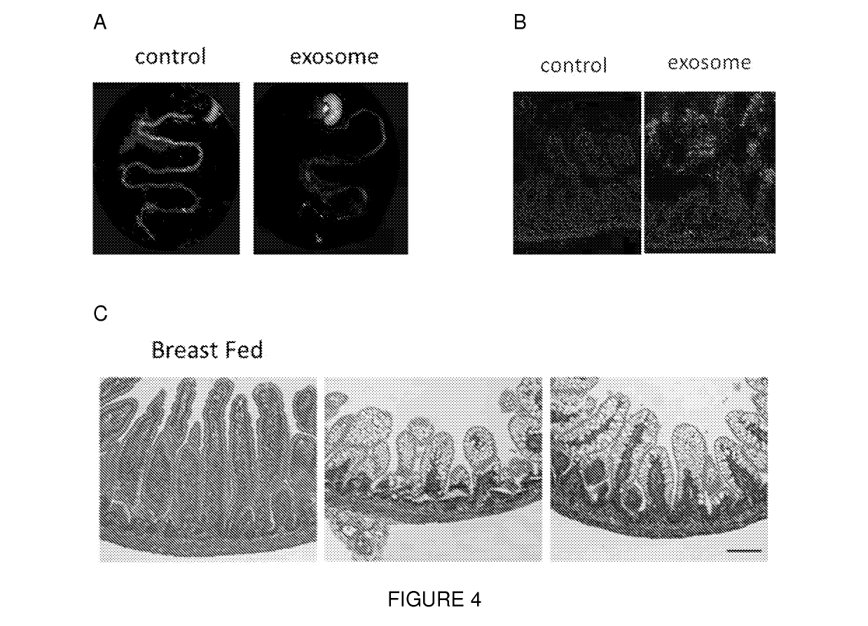

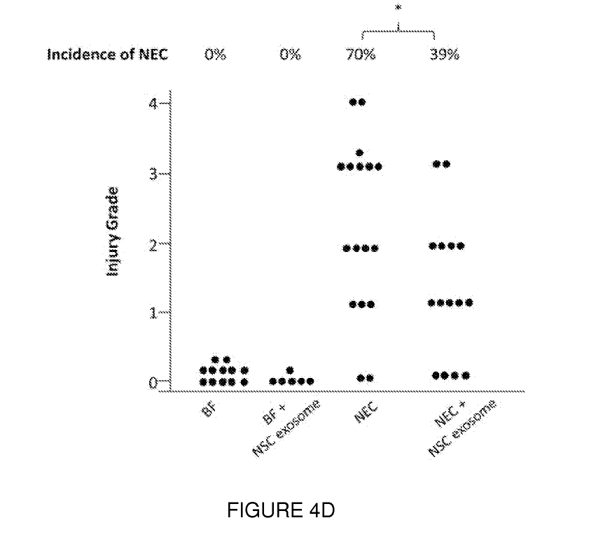

Exosomes were isolated from enteric NSC, stained with PKH26 dye, and applied to a mixed culture of rat intestinal cells 30 minutes prior to anoxia/reoxygenation (A/R) (4 h/24 h) injury. The cells were stained for the mature neuron marker Tuj-1 (green); stained for exosomes (red) and the nuclei were stained with DAPI (blue). Red cytoplasmic staining in target cells confirmed uptake of NSC-derived exosomes in injured neurons as shown in FIG. 4.

Enteric NSC were transfected with pGFP-hHB-EGF and exosomes were harvested 48 hours later. The exosomes were subsequently stained with PKH26 and applied to a mixed culture of rat intestinal cells 30 min prior to A/R (4 h/24 h). The cells were stained as described above for the mature neuron marker Tuj-1 (grey); exosomes (red) HB-EGF (green) and the nuclei were stained with DAPI (blue). As shown in FIG. 4, green staining of exosomes confirms HB-EGF loading; yellow merged cytoplasmic staining in target cells confirms that exosomal HB-EGF was transferred to injured neurons.

The addition of native or HB-EGF-enriched NSC-derived exosomes to mixed cultures of rat intestinal cells exposed to A/R resulted in exosomal localization and HB-EGF delivery to injured enteric neurons.

### Example 6

### SC-Derived Exosomes can Protect the Intestines from NEC

Protective SC were harvested from mice, and exosomes were purified from conditioned medium of the SC using the PureExo Isolation kit and characterized as described in Example 1. Exosomes were stained with the red fluorescent cell linker dye PKH26 to allow their distribution to be tracked. Neonatal mice were exposed to experimental NEC as described in Example 3. Pups were randomly assigned to the following groups: (1) breast feeding only; (2) NEC−no treatment, (3) NEC+PBS, (4) NEC+mesenchymal stem cells (MSC) and (5) NEC+exosomes IP.

Equal numbers of exosomes to be delivered are determined using the small particle detection capabilities of the BD Influx flow Cytometer, calibrated down to 50 nm. Exosomes were administered intraperitoneally 8 hours after the pups were exposed to experimental NEC. Pups were sacrificed upon development of signs of NEC (bloody stools, abdominal distention, respiratory difficulty, lethargy) or at the end of the experiment at 72 hours, and analyzed for specific endpoints related to the ENS, as well as generalized endpoints related to NEC.

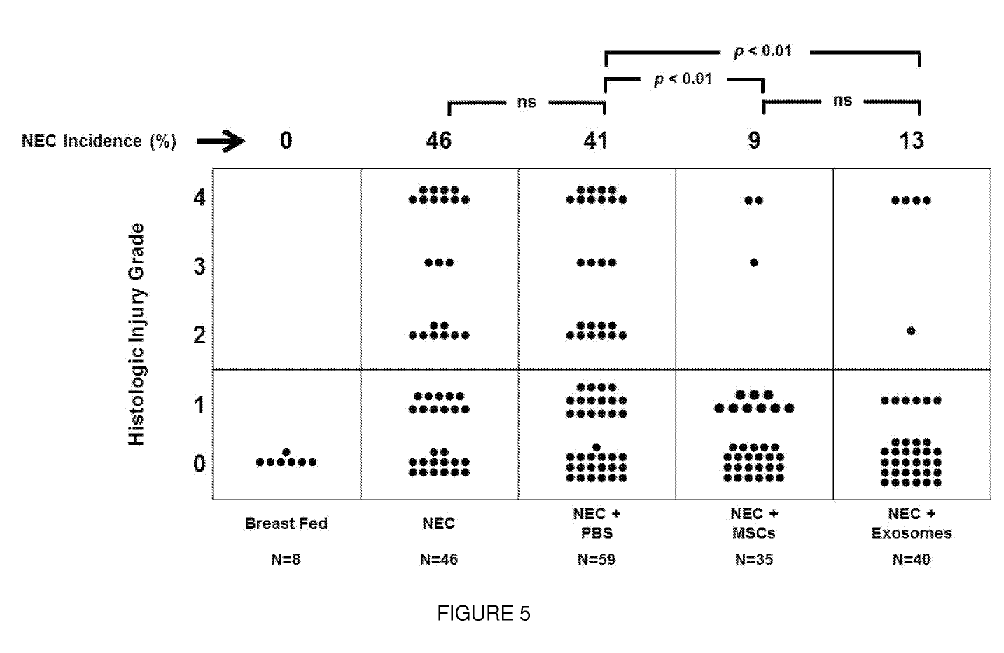

As shown in FIG. 5, the pups that were breast fed were found to have not experimental NEC. The pups which were exposed to only the experimental NEC model has 46% incidence of NEC, and the pups exposed to the experimental NEC model and only received the vehicle (PBS) had 41% NEC. The administration of stem cells and exosomes from those stem cells results in a statistically significant decrease in the incidence of NEC. There was no statistical difference between the incidence of NEC between the control group, nor was there a statistical difference between the treatment groups (MCS or Exosomes).

### Example 7

### Direct Transfer of Exosomes from Donor SC to Recipient Intestinal/ENS Cells Protects Recipient Cells from Injury

A co-culture system comprising silicone micro-culture devices containing two wells (0.22 cm²/well) is used to culture cells that are physically separated from each other by a central silicone wall. One well receives ˜3,000 donor SC which are allowed to adhere for 12 hours, and then the other well is seeded with recipient cells (using cell lines if available or using primary cells purified directly from intestine with immunoaffinity techniques). The silicone wall is then removed to allow secreted exosomes to move from donor to recipient cells. First, donor SC are transfected with pGFP/CD9 to allow exosome-associated CD9 to be tracked. GFP fluorescence in recipient cells is demonstrate that GFP-tagged CD9 transcript and/or protein was transferred from donor to recipient cells. To confirm that exosomes mediate this process, donor cells are pre-treated with GW4869 (0-100 μg/ml), which is a well-characterized exosome inhibitor that blocks neutral sphingomyelinase 2 (nSMAse2), which is required for the biosynthesis of ceramide on which exosome production is dependent or with siRNA to nSMAse2 to block the production of exosomes. To confirm that HB-EGF mRNA is shuttled from donor to recipient cells via exosomes, donor SC is transfected with the pEGFP/hHB-EGF vector (Origene) or mock-transfected. Recipient cells are analyzed by RT-PCR for hHB-EGF using human-specific primers. To demonstrate that the transfer is dependent on exosomes, some wells of donor SC will be pre-treated with GW4868 (0-100 μM) or nSMase si-RNA, either of which will reduce the level of GFP/hHB-EGF transcripts in both exosomes and recipient cells.

To establish that donor SC deliver HB-EGF protein to recipient cells, recipient cells are analyzed by direct fluorescence for GFP, and by ICC/Western blot for GFP or HB-EGF. To determine whether HB-EGF is delivered by exosomes in an already-translated form as protein or is translated in the recipient cells after delivery of the transcript, the following steps are used. First, recipient cells are treated with cyclohexamide to block protein synthesis. Second, recipient cells are pre-treated with pRFP/si-HB-EGF to deplete their endogenous HB-EGF mRNA and protein levels and to block their ability to translate protein from the delivered GFP/hHB-EGF transcript. For cyclohexamide- or si-HB-EGF-treated cells, any HB-EGF or GFP signal in recipient cells are due to direct transfer of GFP/hHB-EGF protein rather than mRNA. Conversely, GFP signal that is lost are attributable to protein translation from delivered GFP/hHB-EGF mRNA. Third, the time course of appearance of GFP/hHB-EGF in recipient cells are more rapid if delivered as protein vs. transcript since the latter must first be translated. Thus, recipient cells are examined hourly over 12 hours for HB-EGF or GFP protein, or for GFP/hHB-EGF transcript. Early HB-EGF and GFP protein signals correspond to delivery of GFP/hHB-EGF protein and are followed several hours later by a wave of HB-EGF and GFP protein signals if the GFP/hHB-EGF transcript is subsequently translated. This analysis demonstrates that that: (i) treatment of the donor SC with nSMase2 si-RNA or GW4869 ablates GFP/hHB-EGF protein in recipient cells thereby proving involvement of exosomes; or (ii) neutralizing anti-HB-EGF IgG added to the medium of donor or recipient cells does not block the appearance of GFP/hHB-EGF protein in recipient cells, thereby ruling out uptake of soluble (secreted) GFP/hHB-EGF by recipient cells. These experiments show that HB-EGF can be transferred from SC to one or more of the recipient cell types as part of a normal exosomal shuttling mechanism between the cells.

To demonstrate that exosomal HB-EGF protects recipient cells from injury, studies are performed on control recipient cells and on recipient cells that were first exposed to anoxia (95% N2/5% CO2) for 4 hours followed by reoxygenation for 24 hours. Exosomes are enriched for HB-EGF by transfection of donor SC with pEGFP/hHB-EGF or pCMV/hHB-EGF and exosomes isolated using PureExo. Exosomes are added to recipient cells for 3, 6, 12, or 24 hours prior to exposure of the cells to anoxia, or at 0, 3, 6, 12, or 24 h after anoxia. To assess their response to injury, recipient cells will be examined for morphological changes, apoptosis as determined by TUNEL and anti-caspase-3 staining, and necrosis as determined by LDH cytotoxicity assay. To confirm that protection of recipient cells is due to transfer of HB-EGF mRNA or protein from donor SC, HB-EGF-depleted exosomes are collected from SC in which HB-EGF has been silenced by si-HB-EGF have a compromised effect on recipient cells. Alternatively, exosomes are purified from donor SC harvested from HB-EGF KO mice. All of these techniques, as well as HB-EGF KO mice, are routine in the art.

### Example 8

### Therapeutic Value of Exosomally-Delivered HB-EGF in Experimental NEC

To load exosomes with HB-EGF, protective SC are transfected with a human HB-EGF plasmid, as we have described (James et al., J Surg Res 2010; 163:86-95; Fagbemi et al. Early Hum Dev 2001; 65:1-9). Exosomes are purified from control and HB-EGF-overexpressing SC using the PureExo kit and stained with the red fluorescent cell linker dye PKH26. Increased levels of HB-EGF mRNA and protein in exosomes from HB-EGF-overexpressing SC are confirmed by RT-PCR and Western blot. Neonatal mice are exposed to experimental NEC as described in Example 3. Pups will be randomly assigned to the following groups: (1) BF; (2) NEC; (3) NEC+control exosomes; and (4) NEC+HB-EGF-loaded exosomes. Exosome quantification is performed using the BD Influx flow Cytometer. Exosomes are delivered by either IP or IV injection immediately after birth or after 24 hours of exposure to stress. Pups are sacrificed upon development of signs of NEC (bloody stools, abdominal distention, respiratory difficulty, lethargy) or at the end of the experiment at 72 hours, and analyzed for specific endpoints related to the ENS and generalized endpoints related to NEC.

What is claimed:

1. A stem cell derived exosome comprising heparin binding epidermal growth factor (HB-EGF) product or a fragment thereof wherein the exosome is produced by a stem cell transfected to express HB-EGF product or a fragment thereof.

2. The stem cell derived exosome of claim 1 wherein the exosome is derived from a neural stem cell.

3. The method of claim 1 or 2, wherein the HB-EGF product comprises amino acids of 74-148 of SEQ ID NO: 2.

4. A composition comprising the exosomes of claim 1 and a carrier.

5. A method of delivering a heparin binding epidermal growth factor (HB-EGF) product or a fragment thereof to a site of intestinal injury comprising administering stem cell-derived exosomes comprising HB-EGF to a subject suffering from an intestinal injury.

6. Use of a stem cell-derived exosome for the preparation of a medicament for the delivery of a heparin binding epidermal growth factor (HB-EGF) product or a fragment thereof to a site of an intestinal injury in a subject in need thereof, wherein the exosome comprises the HB-EGF product or a fragment thereof.

7. A method of treating an intestinal injury comprising administering a stem cell-derived exosome comprising administering a heparin binding epidermal growth factor (HB-EGF) product or a fragment thereof to a subject suffering from an intestinal injury in an amount effective to reduce the severity of the intestinal injury.

8. Use of a stem cell-derived exosome comprising a heparin binding epidermal growth factor (HB-EGF) product or a fragment thereof for the preparation of a medicament for the treatment of an intestinal injury in a subject in need thereof, wherein the cell-derived exosome comprises an amount of HB-EGF product or a fragment thereof effective to reduce the severity of the intestinal injury.

9. A method of reducing damage to intestinal tissue in a subject suffering from an intestinal injury comprising administering a cell-derived exosome comprising a heparin binding epidermal growth factor (HB-EGF) product or a fragment thereof in an amount effective to protect the intestinal tissue in the subject.

10. Use of a stem cell-derived exosome comprising a heparin binding epidermal growth factor (HB-EGF) product or a fragment thereof for the preparation of a medicament for the reduction of damage to intestinal tissue in a subject suffering from an intestinal injury, wherein the cell-derived exosome comprises an amount of HB-EGF.

11. The method or use of any one of claims 5-10 wherein the stem cell is a neural stem cell.

12. The method or use of claim 11 wherein the stem cells are transfected to express HB-EGF or a fragment thereof.

13. The method or use of any one of claims 5-10, wherein the HB-EGF product comprises amino acids of 74-148 of SEQ ID NO: 2.

14. The method or use of any one of claims 5-13, wherein the intestinal injury is caused by necrotizing enterocolitis, hemorrhagic shock, resuscitation, ischemia/reperfusion injury, intestinal inflammatory conditions or intestinal infections.

15. The method or use of any one of claims 5-13, wherein the subject is suffering from Hirschprung's Disease, intestinal dysmotility disorders, intestinal pseudo-obstruction (Ogilvie's Syndrome), inflammatory bowel disease, irritable bowel syndrome, radiation enteritis or chronic constipation, Chrohn's Disease, bowel cancer, or colorectal cancers.

16. The method or use of any one of claims 5-15 wherein the subject is an infant.

17. The method of any one claims 5-16, wherein the exosomes are administered intravenously or intraperitoneally.

18. The method of any one of claim 5-17, wherein the exosomes are administered immediately following the intestinal injury or within 1-5 hours following the intestinal injury.

19. A stem cell-derived exosome comprising a heparin binding epidermal growth factor (HB-EGF) product or a fragment thereof for delivering HB-EGF product or a fragment thereof to a site of intestinal injury in a subject in need thereof.

20. A stem cell-derived exosome for the treatment of an intestinal injury in a subject in need thereof, wherein the stem cell-derived exosome comprises a heparin binding epidermal growth factor (HB-EGF) product or a fragment thereof in an amount effective to reduce the severity of the intestinal injury.

21. A stem cell-derived exosome for the reduction of damage to intestinal tissue in a subject suffering from an intestinal injury, wherein the stem cell-derived exosome comprises a heparin binding epidermal growth factor (HB-EGF) product or a fragment thereof in an amount effective to protect the intestinal tissue in the subject.

22. The stem cell-derived exosome of any one of claims 19-21 wherein the stem cell is a neural stem cell.

23. The stem cell-derived exosome of claim 22 wherein the stem cells are transfected to express HB-EGF or a fragment thereof.

24. The stem cell-derived exosome of any one of claims 19-23, wherein the HB-EGF product comprises amino acids of 74-148 of SEQ ID NO: 2.

25. The stem cell-derived exosome of any one of claims 19-24, wherein the intestinal injury is caused by necrotizing enterocolitis, hemorrhagic shock, resuscitation, ischemia/reperfusion injury, intestinal inflammatory conditions or intestinal infections.

26. The stem cell-derived exosome of any one of claims 19-25, wherein the subject is suffering from Hirschprung's Disease, intestinal dysmotility disorders, intestinal pseudo-obstruction (Ogilvie's Syndrome), inflammatory bowel disease, irritable bowel syndrome, radiation enteritis or chronic constipation, Chrohn's Disease, bowel cancer, or colorectal cancers.

27. The stem cell-derived exosome of any one of claims 19-26, wherein the subject is an infant.

28. The stem cell-derived exosome of any one claims 19-27, wherein the exosomes are administered intravenously or intraperitoneally.

29. The stem cell-derived exosome of any one of claim 19-28, wherein the exosomes are administered immediately following the intestinal injury or within 1-5 hours following the intestinal injury.


SEQUENCE LISTING (16 sequences)

SEQ ID NO: 1 - DNA, 624 bp, Homo sapiens
  CDS (1)..(624)
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccac

SEQ ID NO: 2 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 3 - DNA, 4913 bp, Homo sapiens
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaaaaaaaaaaaaaaaaaaaaaaaaaaaa

SEQ ID NO: 4 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 5 - DNA, 4261 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 6 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 7 - DNA, 1270 bp, Homo sapiens
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaacagaaaaaaaaaaaaaaaaaaaaaaaaaaaaaaaaa

SEQ ID NO: 8 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 9 - DNA, 1323 bp, Homo sapiens
gcccgaatatgtccctgggtgtgggtatgggtgtggggcaatttgggtgggagcagcgtggaggctcccaggaccaagtcctgcgcctctttggcggggtgtgtgcaggaggaggggggataaataggaggctccctcctcccggcgacattcacggagccggccggcctcccgccctgggtgtttccctgccttgtagccagggtgccagcctgggaagtagtttcgtttccttctgcctccgggattagtttccaggcaccctctcaggcgcccgaggcccgggaagggggcgaagaaggagggagacttgtctaggggctgcccggcccggcagagcggggttgatggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctc

SEQ ID NO: 10 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 11 - DNA, 4628 bp, Homo sapiens
tcacttgcctgatatttccagtgtcagagggacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagaatatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggcagggataacagtgtgcctggttaatattaatattcccattttattaataatatttatgttgggtcaagtgttaggtcaataacactgtattttaatgtacttgaaaaatgtttttatttttgttttatttttgacagactatttgctaatgtataatgtgcagaaaatatttaatatcaaaagaaaattgatatttttatacaagtaatttcctgagctaaatgcttcattgaaagcttcaaagtttatatgcctggtgcacagtgcttagaagtaagcaattcccaggtcatagctcaagaattgttagcaaatgacagatttctgtaagcctatatatatagtcaaatcgatttagtaagtatgttttttatgttcctcaaatcagtgataattggtttgactgtaccatggtttgatatgtagttggcaccatggtatcatatattaaaacaataatgcaattagaatttgggagaagcaaatataggtcctgtgttaaacactacacatttgaaacaagctaaccctggggagtctatggtctcttcactcaggtctcagctataattctgttatatgaggggcagtggacagttccctatgccaactcacgactcctacaggtactagtcactcatctaccagattctgcctatgtaaaatgaattgaaaaacaattttctgtaatcttttatttaagtagtgggcatttcatagcttcacaatgttccttttttgtatattacaacatttatgtgaggtaattattgctcaacagacaattagaaaaaagtccacacttgaagcctaaatttgtgctttttaagaatatttttagactatttctttttataggggctttgctgaattctaacattaaatcacagcccaaaatttgatggactaattattattttaaaatatatgaagacaataattctacatgttgtcttaagatggaaatacagttatttcatcttttattcaaggaagttttaactttaatacagctcagtaaatggcttcttctagaatgtaaagttatgtatttaaagttgtatcttgacacaggaaatgggaaaaaacttaaaaattaatatggtgtatttttccaaatgaaaaatctcaattgaaagcttttaaaatgtagaaacttaaacacaccttcctgtggaggctgagatgaaaactagggctcattttcctgacatttgtttattttttggaagagacaaagatttcttctgcactctgagcccataggtctcagagagttaataggagtatttttgggctattgcataaggagccactgctgccaccacttttggattttatgggaggctccttcatcgaatgctaaacctttgagtagagtctccctggatcacataccaggtcagggaggatctgttcttcctctacgtttatcctggcatgtgctagggtaaacgaaggcataataagccatggctgacctctggagcaccaggtgccaggacttgtctccatgtgtatccatgcattatataccctggtgcaatcacacgactgtcatctaaagtcctggccctggcccttactattaggaaaataaacagacaaaaacaagtaaatatatatggtcatatacatattgtatatatattcatatacaaacatgtatgtatacatgaccttaatggatcatagaattgcagtcatttggtgctctgctaaccatttatataaaacttaaaaacaagagaaaagaaaaatcaattagatctaaacagttatttctgtttcctatttaatacagctgaagtcaaaatatgtaagaacacattttaaatactctacttacagttggccctctgtggttagttccacatctgtggattcaaccaaccaaggacggaaaatgcttaaaaaataatacaacaacaacaaaaaatacattataacaactatttacttttttttttttctttttgagatggagtctcgctctgttgcccaggttggagtgcagtggcacgatctcggctcactgcaacctcacctcccgggttcaagagatcctcctgcctcagcctcctgagcagctgggactacaggcgcatgccaccatgcccagctaatttttgtatttttagtagaggcggggtttcaccatgttggccaggatggtctcaatctcctaaccttgagatccaccctccacagcctcccaaactgctgggattacaggtgtgagccaccgcacgtagcatttacattaggtattacaagtaatgtaaagatgatttaagtatacaggaggatgtgaataggttatatgcaagcactatgcccttttatataagtgacttgaacatctgtgcccgattttagtatgtgcaggggggcgatctgggaatcagtcccctgtggataccaaggtacaactgtatttattaacgcttactagatgtgaggagagtctgaatattttcagtgatcttggctgtttcaaaaaaatctattgacttttcaataaatcagctgcaatccatttatttcatttacaaaagatttattgtaagcatctcaatcttggtttgtcagtttatcttaagcatgtcaattcataaaaacaagtcatttttgtatttttcatctttaagaatgcttaaaaaagctaatccctaaaatagttagatctttgtaaatgcatattaaataataaagtatgacccacattactttttatgggtgaaaataagacaaaaataatagttttagtgaggatggtgctgagtaaacataaaaactgatttgctctcagctgatgtgtcctgtacacagtgggaagattttagttcacacttagtctaactcccccattttacagatttctcactatatatatttctagaaggggctatgcatattcaatgtattgagaaccaaagcaaccacaaatgcataaatgcataatttatggtcttcaaccaaggccacataataacccagttaacttactctttaaccaggaatattaagttctataactagtactcaaggtttaaccttaaaattaagatttccttaaccttaaccttaaaattgatattatattaaacatacataatacaatgtaactccactgttctcctgaatattttttgctctaatctctctgccgaaagtcaaagtgatgggagaattggtatactggtatgactacgtcttaagtcagatttttatttatgagtctttgagactaaattcaatcaccaccaggtatcaaatcaacttttatgcagcaaatatatgattctagtgtctgacttttgttaaattcagtaatgcagtttttaaaaacctgtatctgacccactttgtaatttttgctccaatatccattctgtagacttttgaaaaaaaagtttttaatttgatgcccaatatattctgaccgttaaaaaattcttgttcatatgggagaagggggagtaatgacttgtacaaacagtatttctggtgtatattttaatgtttttaaaaagagtaatttcatttaaatatctgttattcaaatttgatgatgttaaatgtaatataatgtattttctttttattttgcactctgtaattgcactttttaagtttgaagagccattttggtaaacggtttttattaaagatgctatggaacataaagttgtattgcatgcaatttgaagtaacttatttgactatgaatgttatcggattactgaattgtatcaatttgtttgtgttcaatatcagctttgataattgtgtaccttaagatattgaaggagaaaatagataatttacaagatattattaatttttatttatttttcttgggaattgaaaaaaattgaaataaataaaaatgcattgaacatcttgcattcaaaatcttcactgac

SEQ ID NO: 12 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 13 - DNA, 847 bp, Homo sapiens
cgtcagtctagaaggataagagaaagaaagttaagcaactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtatgaaaaagacaaaatatgaagtcacttcatatgcaatcgtttgacaaatagttattcaggccctataatgtgtcaggcactgacatgtaaaatttttttaattaaaaaagagctgtaatctggcaaaaagtttctatgtaatatttttcatgccttttctcataaacccagacgagtggtaaaaatttgccttcagttgtaataggagagttcaaacgtacagtctcccttcaacctatctctgtctgcccatatcaaaattataaatgaggaggacagcaggccccaagaaagtagggactaagtatgtcttgttcaaaattgtatattcagtgacttacactatgcctagcacacaacacacactgagtaaatatttgttgagtgaaataaaatcaagaaacaagtaaaaactga

SEQ ID NO: 14 - protein, 133 aa, Homo sapiens
MALGVPISVYLLFNAMTALTEEAAVTVTPPITAQQADNIEGPIALKFSHLCLEDHNSYCINGACAFHHELEKAICRCFTGYTGERCEHLTLTSYAVDSYEKYIAIGIGVGLLLSGFLVIFYCYIRKRYEKDKI

SEQ ID NO: 15 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 16 - protein, 1210 aa, Homo sapiens [sequence omitted]